(19) United States
(12) Patent Application Publication
(10) Pub. No.: US 2022/0050829 A1
(43) Pub. Date: Feb. 17, 2022

(54) DATA AGGREGATION SYSTEM

(71) Applicant: The Power Player Inc., Buffalo, NY (US)

(72) Inventors: David E. MASON, Barrington, IL (US); Kevin KRUEGER, Bay Village, OH (US); Steven M. KULL, Hamburg, NY (US); Brendan J. STAUB, Grand Island, NY (US); Joseph P. GANNON, Buffalo, NY (US)

(21) Appl. No.: 17/513,792

(22) Filed: Oct. 28, 2021

Related U.S. Application Data
(63) Continuation of application No. 16/167,428, filed on Oct. 22, 2018.
Application No. 16/167,428 is a continuation of application No. 15/169,604, filed on May 31, 2016, now abandoned.
(60) Provisional application No. 62/168,776, filed on May 30, 2015.

(51) Int. Cl. G06F 16/242; G06Q 10/06; G01D 9/00; G06F 16/2455; G06F 16/2457; G16B 40/00

(52) U.S. Cl. CPC G06F 16/244; G06Q 10/063; G16B 40/00; G06F 16/24556; G06F 16/24578; G01D 9/005

(57) ABSTRACT

A method and a system for data aggregation for human beings is a single point for collection, aggregation, visualization, and selective distribution of quantitative and qualitative data. The quantitative and qualitative data pertains to various domains which include sports, education, music, healthcare, animal data and the like. The system provides development tools and assessment tools for each human being. The system also includes facilitating the plurality of human beings and a plurality of respective stake-holders to enter qualitative and quantitative information on a web-based platform, collecting the qualitative and quantitative information, analyzing the qualitative and quantitative information, aggregating and visualizing the qualitative and quantitative information, and selectively distributing the qualitative and quantitative information to the plurality of human beings and the plurality of stake-holders.

### CROSS-REFERENCE TO RELATED APPLICATIONS

This application is a continuation of U.S. patent application Ser. No. 16/167,428, filed Oct. 22, 2018 and entitled “DATA AGGREGATION SYSTEM”, which application is a continuation of U.S. patent application Ser. No. 15/169,604, filed May 31, 2016 and entitled “DATA AGGREGATION SYSTEM”, which application claims the benefit under 35 U.S.C. § 119(e) of U.S. Provisional Patent Application Ser. No. 62/168,776, filed May 30, 2015, entitled “DATA AGGREGATION SYSTEM”, the disclosures of which are herein incorporated by reference.

### TECHNICAL FIELD

The present invention relates generally to data aggregation systems and methods and, more particularly, to the data aggregation systems and methods for human beings, animals and plants.

### BACKGROUND

During the last several years, the advancements in technology have had a tremendous impact on lives of all living creatures. There are various real life scenarios where use of internet, electronics and communication devices such as personal computers, sensing networks and devices, hand-held personal organizers and computerized automotive controls have changed one's daily life. Most of technologies have made performing the task easier for human beings rather than helping them with their decision-making.

In the present scenario, a person, or technology user, has to make various decisions associated with his/her profession, career and other decisions associated with various other entities such as animals belonging to them or owned by them, animals in vicinity of their area or place and the like. However, these decisions taken by technology users, or users, may backfire on them as these decisions are merely based on a personal view or judgment. More often than not, the users do not have an aggregated data of various activities performed by him over a past few years, his likes and dislikes, personal interests and the like. Further, as animals have become an important part in the lives of the humans, it is a common trend to see a person owning and raising an animal. More often than not, the humans have a tendency to ignore value and importance of taking decisions for the animals owned by them. Moreover, the humans do not have aggregated data associated with one or more animals owned by them or the one or more animals living in the vicinity. Furthermore, it is important for users to have detailed information about the animals which they want to buy or the animals which are located in their area.

In addition, various stake-holders (for example, guardians) play an important role in assisting their children (the users) on various fundamental decisions. The stake-holders do not have a single point of viewing and analyzing qualitative and quantitative data.

In light of the above stated discussion, there is a need for a method and system which provide a single point for collection, aggregation, visualization and selective distribution of the quantitative and the qualitative data.

### SUMMARY

The present invention aims to overcome the above mentioned limitations and other problems associated with the prior art.

The present disclosure provides a method and a system for data aggregation for human beings, animals and plants in general. The data aggregation system is a single point for collection, aggregation, visualization, and selective distribution of quantitative and qualitative data. The data aggregation system provides development tools and assessment tools. The quantitative and qualitative data pertains to various domains which include sports, education, music, healthcare, animals and the like. The data aggregation system includes facilitating the plurality of users and a plurality of stake-holders to enter the qualitative and quantitative data on a web-based platform, collecting the qualitative and quantitative data, analyzing the qualitative and quantitative data, aggregating and visualizing the qualitative and quantitative data, and selectively distributing the qualitative and quantitative data to the plurality of users and the plurality of stake-holders.

In an embodiment of the present disclosure, the data aggregation system enables the plurality of stake-holders to provide specific comments and suggestions to the plurality of users.

In an embodiment of the present disclosure, the data aggregation system provides a chance to the plurality of users to earn profits based on their ratings.

In an embodiment of the present disclosure, the data aggregation system provides motivation and creates community and connection for the plurality of users and associated stake-holders.

In an embodiment of the present disclosure, the data aggregation system provides a feedback to the plurality of users and the associated stake-holders.

In an embodiment of the present disclosure, the data aggregation system provides parameters for measuring strengths, weakness and progress of each of the plurality of users and the associated stake-holders.

The data aggregation system could be designed to work with various types of data, such as date pertinent to athletes, coaches-players, students, teachers-students, and animals, such as dogs or horses who are being trained by human trainers.

### BRIEF DESCRIPTION OF THE FIGURES

Having thus described the invention in general terms, reference will now be made to the accompanying drawings, which are not necessarily drawn to scale, and wherein:

FIG. 1 illustrates a data aggregation system, in accordance with various embodiments of the present disclosure;

FIG. 2 illustrates the data aggregation system for aggregating animal related data, in accordance with an embodiment of the present disclosure;

FIG. 3A illustrates the data aggregation system for aggregating sports related data, in accordance with an embodiment of the present disclosure;

FIG. 3B illustrates the data aggregation system for aggregating health related data, in accordance with another embodiment of the present disclosure;

FIG. 3C illustrates the data aggregation system for aggregating education related data, in accordance with yet another embodiment of the present disclosure;

FIG. 3D illustrates the data aggregation system for aggregating music related data, in accordance with yet another embodiment of the present disclosure;

FIG. 3E illustrates the data aggregation system for aggregating miscellaneous data corresponding to multiple users, in accordance with yet another embodiment of the present disclosure;

FIG. 4 illustrates a block diagram of the data aggregation system, in accordance with various embodiments of the present disclosure; and

FIG. 5 provides a brief general description of a suitable computing environment in which the disclosure can be implemented.

### DETAILED DESCRIPTION

The exemplary embodiments described herein for illustrative purposes are subject to many variations in structure and design. It should be emphasize, however, that the present invention is not limited to method for erasing data from a volatile memory. It is understood that various omissions and substitutions of equivalents are contemplated as circumstances may suggest or render expedient, but these are intended to cover the application or implementation without departing from the spirit or scope of the present invention.

The terms “a” and “an” herein do not denote a limitation of quantity, but rather denote the presence of at least one of the referenced item.

The terms “having”, “comprising”, “including”, and variations thereof signify the presence of a component.

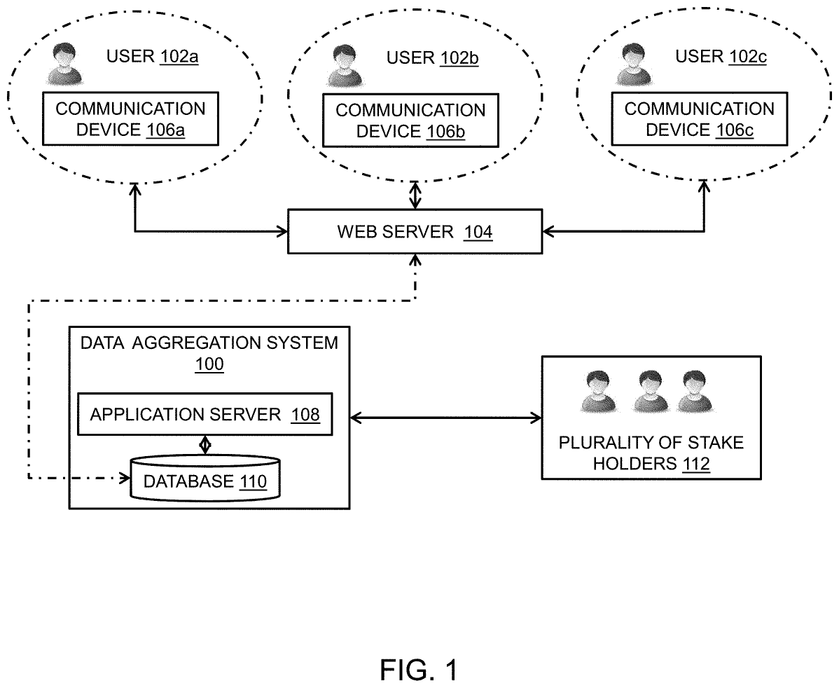

FIG. 1 illustrates a data aggregation system 100, in accordance with various embodiments of the present disclosure. The data aggregation system 100 is a single point for collection, aggregation, visualization and selective distribution of human related quantitative and qualitative data. The aggregation, visualization and selective distribution of the quantitative and the qualitative human related data empower multiple stake-holders to take informed decisions at different stages. In addition, the data aggregation system 100 provides development tools, assessment tools for the users, and keeps the users engaged not only with each other but also with various stake-holders.

As shown in FIG. 1, a user 102a, a user 102b and a user 102c interacts with the data aggregation system 100 through a web server 104. The user 102a is associated with a communication device 106a, the user 102b is associated with a communication device 106b and the user 102c is associated with a communication device 106c. In an embodiment of the present disclosure, each of the communication devices 106a-c is a portable device. Examples of the communication devices 106a-c include but may not be limited to mobile phones, tablets, laptops and the like. Further, each of the communication devices 106a-c is associated with the web server 104. In simpler terms, each of the plurality of users 102a-c communicates with the web server 104 through the corresponding communication devices 106a-c.

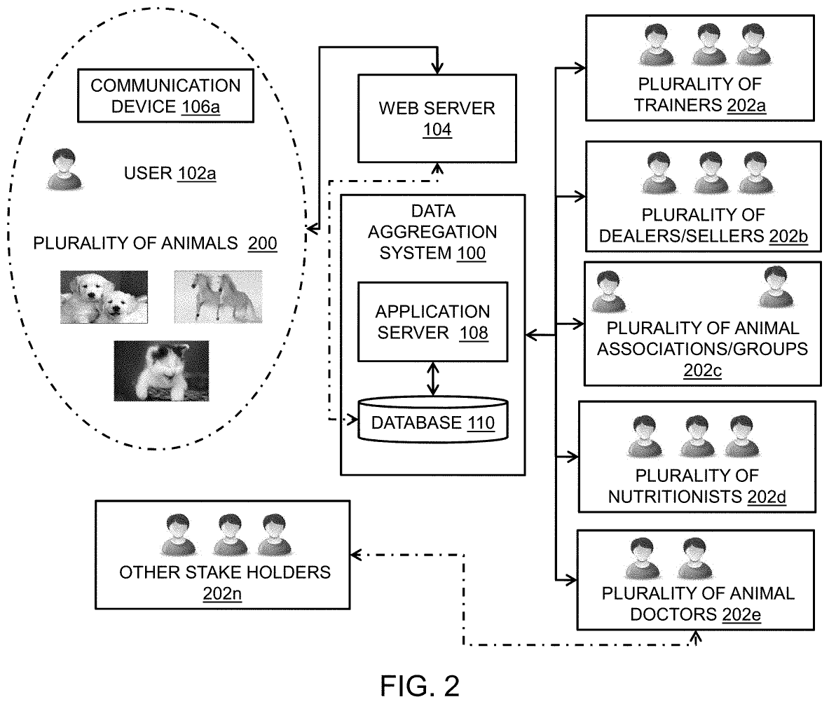

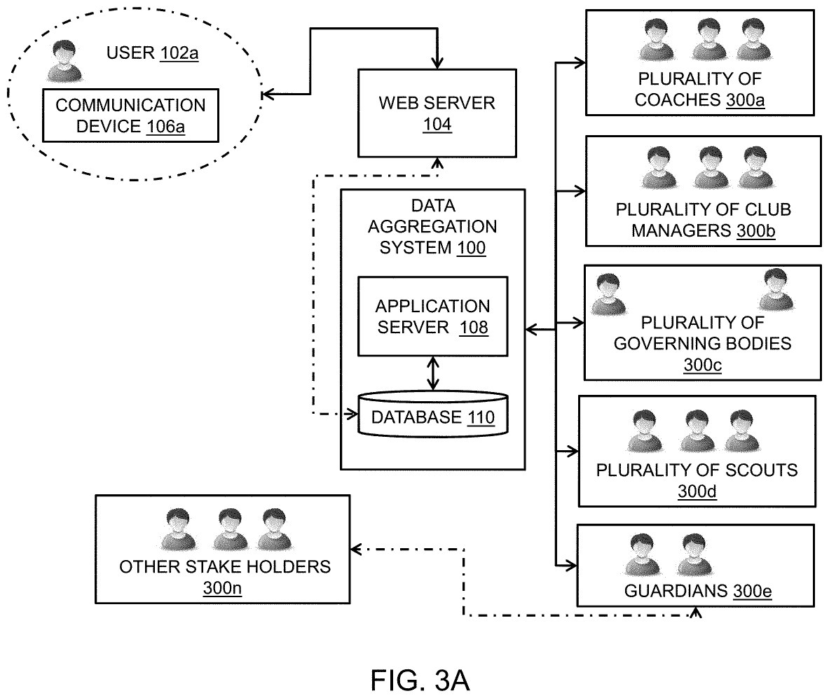

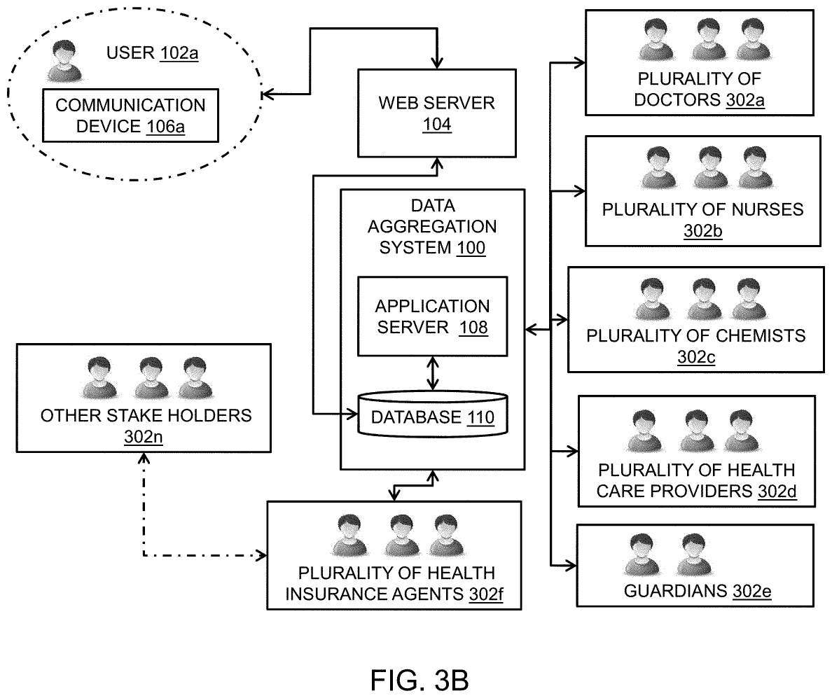

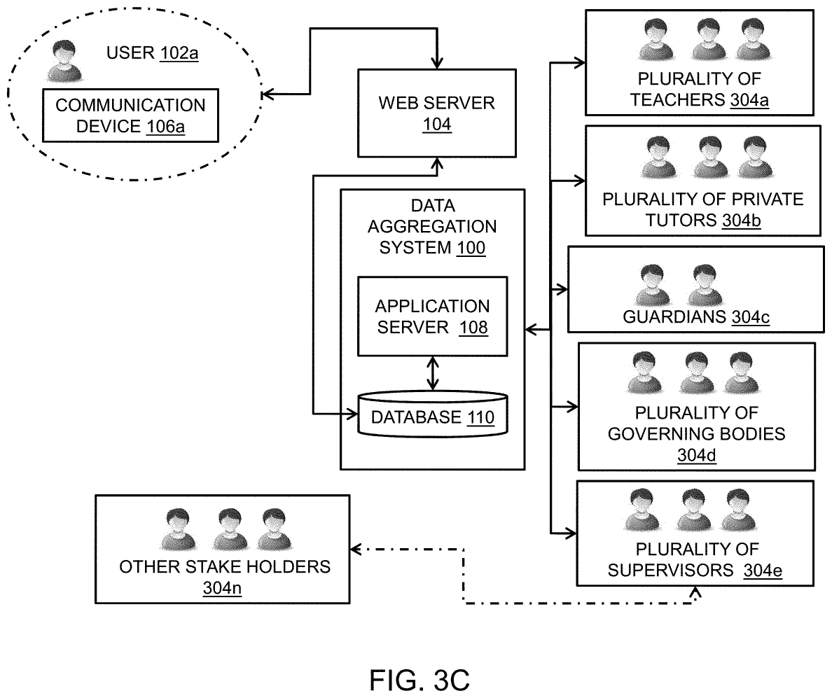

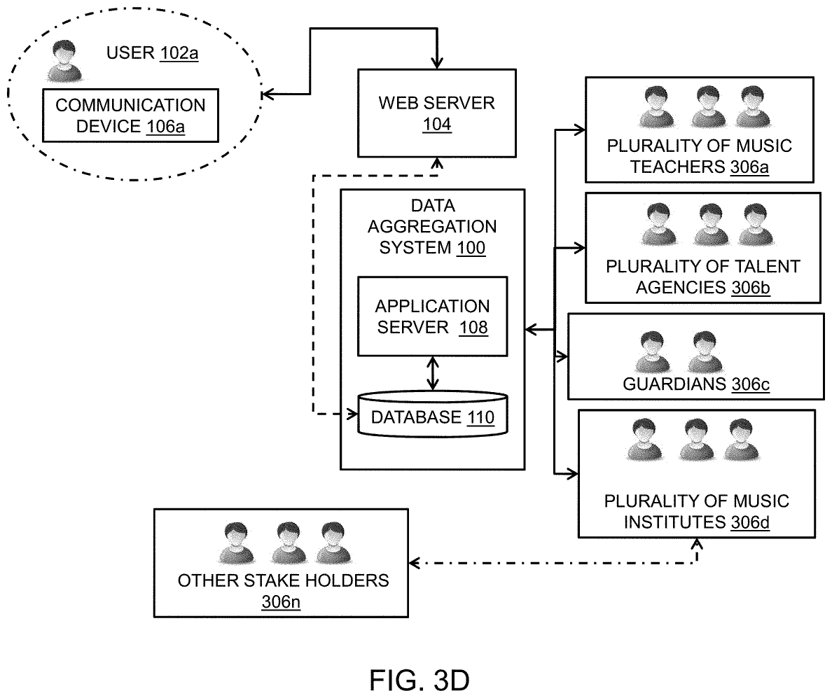

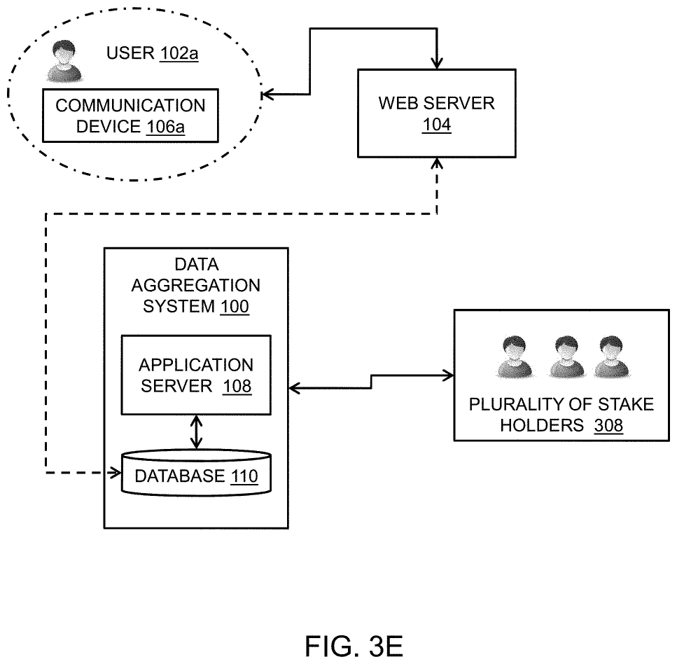

In addition, a plurality of stake-holders 112 is associated with the data aggregation system 100. The plurality of stake-holders 112 include one or more individuals who are related to the users 102a-c (described elaborately in detailed description of FIG. 2, FIG. 3A, FIG. 3B, FIG. 3C, FIG. 3D and FIG. 3E). Going further, each of the plurality of stake-holders 112 interacts with the data aggregation system 100 with a corresponding communication device. The data aggregation system 100 includes an application server 108 and a database 110.

In an embodiment of the present disclosure, the data aggregation system 100 enables the plurality of users 102a-c to register on a web-based platform and/or application supported and/or maintained by the data aggregation system 100 through the corresponding communication devices 106a-c. Further, the data aggregation system 100 enables the plurality of stake-holders 112 to register on the web-based platform and/or application. In an embodiment of the present disclosure, each of the plurality of users 102a-c accesses the web-based platform and/or application through the web server 104. Further, the application server 108 runs the web-based platform and/or application.

Further, the data aggregation system 100 enables the plurality of users 102a-c to enter a first set of quantitative and qualitative information and a second set of quantitative and qualitative information on the web-based platform and/or application.

The first set of quantitative and qualitative information includes human related data (as elaborately described in FIG. 3A, FIG. 3B, FIG. 3C, FIG. 3D and FIG. 3E). The second set of quantitative and qualitative information includes animal related data (as elaborately described in FIG. 2). In an embodiment of the present disclosure, the data aggregation system 100 enables the plurality of users 102a-c to enter the first set of quantitative and qualitative information and the second set of quantitative and qualitative information without registering on the web-based platform and/or the application. In another embodiment of the present disclosure, the data aggregation system 100 enables the plurality of users 102a-c to enter the first set of quantitative and qualitative information and the second set of quantitative and qualitative information by registering on the web-based platform and/or the application.

On the same lines, the data aggregation system 100 enables the stake-holders 112 to enter the first set of quantitative and qualitative information and the second set of quantitative and qualitative information on the web-based platform and/or the application. In another embodiment, the data aggregation system 100 enables the stake-holders 112 to enter their own quantitative and qualitative information on the web-based platform and/or the application.

In an embodiment of the present disclosure, the data aggregation system 100 performs a set of operations based on the first set of quantitative and qualitative information and the second set of quantitative and qualitative information entered by the plurality of users 102a-c. In another embodiment of the present disclosure, the data aggregation system 100 performs a set of operations based on the first set of quantitative and qualitative information and the second set of quantitative and qualitative information entered by the plurality of users 102a-c and by the stake-holders 112. In an embodiment of the present disclosure, the data aggregation system 100 enables the stake-holders 112 to enter the first set of quantitative and qualitative information and the second set of quantitative and qualitative information without registering on the web-based platform and/or application. In another embodiment of the present disclosure, the data aggregation system 100 enables the stake-holders 112 to enter the first set of quantitative and qualitative information and the second set of quantitative and qualitative information by registering on the web-based platform and/or application.

Further, the data aggregation system 100 facilitates storing the information and results of the set of operations in the database 110. Moreover, the plurality of stake-holders 112 may have access to all or subset of the first set of quantitative and qualitative information and the second set of quantitative and qualitative information of each of the plurality of users 102a-c and results of the set of operations performed on the first set of quantitative and qualitative information and the second set of quantitative and qualitative information pertaining to the plurality of users 102a-c. In an embodiment of the present disclosure, the quantitative and qualitative information may be aggregated and selective distributed to the stake-holders 112 (described later in the application). In an embodiment of the present disclosure, the data aggregation system 100 charges an amount from the plurality of users 102a-c and the plurality of stake-holders 112 for establishing and maintaining the account on the web-based platform.

The amount can be payable by at least one of following means including online banking, using credit cards, using debit cards and the like. Furthermore, the data aggregation system 100 generates an id for each of the users 102a-c and each of the stake-holders 112 after receiving the payment from each of the users 102a-c and each of the stake-holders 112. The id enables the users 102a-c and the stake-holders 112 to access the web-based platform. Furthermore, it may be noted that in FIG. 1, three users (102a, 102b and 102c) are shown to interact with the data aggregation system 100; however, those skilled in the art would appreciate that more or lesser number of users can interact with the data aggregation system 100.

FIG. 2 illustrates the data aggregation system 100 for aggregating animal related data, in accordance with an embodiment of the present disclosure. It may be noted that to explain the system elements of FIG. 2, references will be made to the system elements of the FIG. 1.

As shown in FIG. 2, the user 102a interacts with the data aggregation system 100 through a web server 104. The user 102a is associated with the communication device 106a. Further, the communication device 106a is associated with the web server 104. In simpler terms, the user 102a communicates with the web server 104 through the communication device 106a. In addition, the user 102a is associated with a plurality of animals 200. The plurality of animals 200 may include but not be limited to dogs, cats, horses and fishes. In an embodiment of the present disclosure, the plurality of animals 200 includes pets of the user 102a and associated stake holders (described below). In another embodiment of the present disclosure, the plurality of animals 200 includes animals living in the vicinity of user 102a and associated stake holders (described later). In yet another embodiment of the present disclosure, the plurality of animals 200 includes animals living in city/country of user 102a and associated stake holders (described below).

In addition, a plurality of stake-holders 202a-n is associated with the data aggregation system 100. The plurality of stake-holders 202a-n includes a plurality of trainers 202a, a plurality of dealers/sellers 202b, a plurality of animal associations/groups 202c, a plurality of nutritionists 202d, a plurality of animal doctors 202e and other stake-holders 202n. Each of the plurality of stake-holders 202a-n interacts with the data aggregation system 100 with their respective communication devices.

The data aggregation system 100 enables the registration of the user 102a on the web-based platform and/or application. Further, the data aggregation system 100 enables registration of each of the plurality of stake-holders 202a-n on the web-based platform and/or application. In an embodiment of the present disclosure, the data aggregation system 100 enables the user 102a and the stake-holders 202a-n to set up an account on the web-based platform. For setting the account, the user 102a and the plurality of the stake-holders 202a-n enters a set of information on the web-based platform and/or application. The information includes but may not be limited to personal information. The personal information includes a first name, a last name, an email id, a password, a phone number, an address, a date of birth, a city name, a state name, a country name, academic information, family background and the like. The user 102a and the plurality of the stake-holders 202a-n get registered by entering the personal information on the web-based platform.

Further, the data aggregation system 100 enables the user 102a to enter profile information. The profile information includes the second quantitative data and qualitative information. The second quantitative data includes input metrics, uploaded audios/videos and the like (described later). The second qualitative data includes input ratings, points, comments, messages and the like (described later). In an embodiment of the present disclosure, the user 102a enters name of the animal, type of the animal, breed of the animal, animal food provided, animal age, different competitions the animal of the plurality of animals 200 has participated in, animal color, animal weight, training schedules, number of animals owned by the user 102a, name of animal dealer/seller, address of animal dealer/seller, vaccination details and schedules, any disease that the animal had or is having and the like. It may be noted that the scope of the invention is not limited by entering the above stated information as profile information; those skilled in the art would appreciate that the user 102a may enter other related information as well.

In an embodiment of the present disclosure, the plurality of stake holders 202a-n may enter the above stated information on behalf of the user 102a on the web-based platform.

For example, a user X owns a dog Y. The user X enters an information including name (Marley), breed (Labrador), age (10 years), weight (30 lbs), food given (proteins and carbohydrates), doctor name (Dr. Ted Jones) and the like.

In an embodiment of the present disclosure, the plurality of trainers 202a enters the information including a type of animal trained so far, experience, information of their personal animals and the like.

In yet another embodiment, the user 102a may upload demonstration images, audios and videos, demonstration audios and videos, drill schematics and the like. It may be noted that the scope of the invention is not limited by entering the above stated information as the profile information; those skilled in the art would appreciate that the user 102a may enter other related information as well. For example, the user X uploads videos of performances of his dog Y in different dog competitions. In an embodiment of the present disclosure, the guardians of the user 102a may upload these demonstration images, the audios and videos, the demonstration audios and videos, the drill schematics, the article links, and the like on behalf of their children (the user 102a) on the web-based platform. In another embodiment, the plurality of trainers 202a may upload the audios and videos on behalf of the user 102a on the web-based platform. In yet another embodiment, the plurality of animal association/groups 202c may upload the audios and videos on behalf of the user 102a on the web-based platform.

In an embodiment of the present disclosure, the user 102a and the plurality of stake-holders 202a-n may upload the video or image directly from their respective communication devices. In another embodiment of the present disclosure the user 102a and the plurality of stake-holders 202a-n may upload the video or image from a library. The video or image may be pre-uploaded in another website associated with the user 102a and a trainer.

In an embodiment of the present disclosure, the users 102a may give ratings and/or points to the plurality of animals 200 based upon their performances in different animal competitions. In another embodiment of the present disclosure, the users 102a may give ratings and/or points to himself based upon how he takes care of the plurality of animals 200. In yet another embodiment, the plurality of trainers 202a may give the ratings, the points, the comments, the messages to the plurality of animals 200 based upon their performances in different animal competitions. In yet another embodiment, the plurality of stake-holders 202a-n may give the ratings, the points, the comments, the messages to the plurality of animals 200 based upon his performance in different animal competitions. In yet another embodiment, the user 102a may/may not rate a stake holder of the plurality of stake holders 202a-n based on training/food recommended for the plurality of animals 200 and its effect on the plurality of animals 200.

Further, the guardians of the user 102a may provide feedback regarding each of the plurality of trainers 202a, the plurality of animal associations/groups 202c, the plurality of nutritionists 202d and the plurality of animal doctors 202e. In an embodiment of the present disclosure, the guardians may give comments to the above mentioned stake holders. For example, a guardian G1 thinks that under a trainer T1, the condition of their animal (say, a horse) has gone below a suitable mark, and thus rates the trainer T1 very low. On the other hand, the same guardian G1 thinks that under a trainer T2, the condition of their animal (say, the horse) has improved, and thus rates the trainer T2 very high. In yet another embodiment, the user 102a may file a complaint against any doctor of the plurality of doctors 202a.

Further, in an embodiment of the present disclosure, the user 102a and the plurality of stake-holders 202a-n may give the ratings by simply writing one or more values. In another embodiment, the user 102a and the plurality of stake-holders 202a-n may give ratings by choosing from a drop down list (say, a list representing poor, average, good, very good and excellent). In yet another embodiment, the user 102a and the plurality of stake-holders 202a-n may give the ratings by using one or more scales. The one or more scales may feature different skills of the user 102a. In yet another embodiment, the guardians may give comments to the user 102a based on their performances. It may be noted that various rating tools (described above) are provided; however those skilled in the art would appreciate that more rating tools may be provided to rate the users 102a.

In an embodiment of the present disclosure, the user 102a may have the permissions to view other user's profile. Further, the user 102a may provide the ratings and the comments to each other's animals.

Further, the data aggregation system 100 collects the information and the data described above and stores it in the database 110. Further, the data aggregation system 100 collects and maintains a repository of the collected information in the database 110. In an embodiment of the present disclosure, the user 102a may redeem the points obtained/earned by them. In another embodiment of the present disclosure, the user 102a may redeem the points obtained/earned by the plurality of animals 200 owned by him/her. In an embodiment of the present disclosure, the data aggregation system 100 collects the second quantitative and qualitative information from third party sources such as web, third party database, or any other source.

Further, the data aggregation system 100 enables an automatic update of the stored information in the database 110 whenever any additional information is entered by the user 102a and the stake-holders 202a-n. In simpler terms, the database 110 gets updated at regular intervals of time with additional information entered by the user 102a and the stake-holders 202a-n. In an embodiment of the present disclosure, previously stored records are also maintained for future references. In other words, the database 110 is dynamic and updates on periodic basis.

Further, the data aggregation system 100 analyzes the second quantitative and the qualitative data uploaded and/or entered by the user 102a. In an embodiment of the present disclosure, the data aggregation system 100 analyzes the medical condition of the animal of the plurality of animals 200 in a period of time, say, in a month, in a year, and the like. In another embodiment, the data aggregation system 100 analyzes medical condition and fitness of the animal of the plurality of animals 200 under a specific trainer and/or doctor and condition/fitness of the animal affected due to changes in the diet plan and the like.

Continuing with the above stated example, the data aggregation system 100 analyzes that the fitness of the dog Y has improved under guidance of his new trainer rather than his previous trainer. Further, the data aggregation system 100 analyzes that the fitness of the dog Y has improved in last 3 months by changing his diet plan.

The data aggregation system 100 aggregates the analyzed information at one place. The analyzed information includes analysis of the second set of quantitative and the qualitative data. Further, the data aggregation system 100 stores the aggregated information in the database 110. The aggregated information includes a collective data that influences the fitness of the plurality of animals 102a. For example, a data (say, a change in diet plans, and the like) representing a positive impact on the fitness of the dog Y is aggregated at a place and a data that leads to a negative impact on the fitness of the dog Y is aggregated at another place.

In an embodiment of the present disclosure, the data aggregation system 100 aggregates fitness/medical condition of the plurality of animals 200 over a period of time, say a month, a month, a year and the like. In another embodiment, the data aggregation system 100 aggregates the fitness/medical condition of the plurality of animals 200 under a trainer and the plurality of trainers 202a.

Further, the data aggregation system 100 generates and/or maintains pictorial representations and reports based on the analyzed and the aggregated information. The analyzed and the aggregated information can be pictorially represented as mountains, spiders, graphs, pie charts and the like. In an embodiment of the present disclosure, the pictorial representation illustrates the fitness/medical condition of the plurality of animals 200 in a month, or in a year. In another embodiment, the pictorial representation illustrates skills of the plurality of animals 200 in an animal competition or various animal competitions. Further, the pictorial representations may help the user 102a and the plurality of stake-holders 202a-n to further give the ratings, the comments, the points and the like.

In an embodiment of the present disclosure, the data aggregation system 100 enables displaying of a set of the second set of quantitative and qualitative information, the analyzed quantitative and qualitative information, and the aggregated quantitative and qualitative information to each of the plurality of stake-holders 202a-n. For example, the guardians will be able to view one set of the second set of the quantitative and qualitative information, the analyzed quantitative and qualitative information and the aggregated quantitative and qualitative information of the plurality of animals 200. The plurality of trainers 202a and the plurality of animal doctors 202e will be able to view another set of the second set of quantitative and qualitative information, the analyzed quantitative and qualitative information and the aggregated quantitative and qualitative information.

In an embodiment of the present disclosure, an administrator of the data aggregation system 100 sets policies and permission for accessing the information for the user 102a and the plurality of stake-holders 202a-n. In another embodiment, the data aggregation system 100 enables the user 102a to restrict the displaying and accessing of the set of information, the profile information, the analyzed and the aggregated information to the plurality of stake-holders 202a-n. In yet another embodiment, the data aggregation system 100 enables the user 102a to restrict and/or display set of quantitative and qualitative information, the analyzed quantitative and qualitative information and the aggregated quantitative and qualitative information to the plurality of stake-holders 202a-n.

In an embodiment of the present disclosure, the data aggregation system 100 enables the user 102a to set a privacy of his account. In an embodiment of the present disclosure, the data aggregation system 100 recommends the user 102a to provide a protein rich diet to the plurality of animals 200. In another embodiment of the present disclosure, the data aggregation system 100 recommends the user 102a to take out the plurality of animals 200 for a walk daily. In yet another embodiment of the present disclosure, the data aggregation system 100 recommends the plurality of trainers 202a to follow a specific guideline for improving the fitness and the medical condition of the plurality of animals 200.

The recommendations are provided based on the analyzed information, the aggregated information, and the pictorial representations. More specifically, the data aggregation system 100 distributes the quantitative data, the qualitative data, the visualized reports, the pictorial representations and the like to the user 102a and the plurality of stake-holders 202a-n. Moreover, the recommendations prove to be useful for the plurality of animal associations/groups 202c while maintaining data of healthy animal species living in their area.

It may be noted that in FIG. 2, a user 102a interacts with the data aggregation system 100, however those skilled in the art would appreciate that more number of users may interact with the data aggregation system 100.

FIG. 3A illustrates the data aggregation system 100 for aggregating human sports related data, in accordance with an embodiment of the present disclosure. It may be noted that to explain the system elements of FIG. 3A, references will be made to the system elements of the FIG. 1.

As shown in FIG. 3A, the user 102a interacts with the data aggregation system 100 through a web server 104. The user 102a is associated with the communication device 106a. Further, the communication device 106a is associated with the web server 104. In simpler terms, the user 102a communicates with the web server 104 through the communication device 106a.

In addition, a plurality of stake-holders 300a-n is associated with the data aggregation system 100. The plurality of stake-holders 300a-n includes a plurality of coaches 300a, a plurality of club managers 300b, a plurality of governing bodies 300c, a plurality of scouts 300d, guardians 300e and other stake-holders 300n. Each of the plurality of stake-holders 300a-n interacts with the data aggregation system 100 with their respective communication devices.

The data aggregation system 100 enables the registration of the user 102a on the web-based platform and/or application. Further, the data aggregation system 100 enables registration of each of the plurality of stake-holders 300a-n on the web-based platform and/or application. In an embodiment of the present disclosure, the data aggregation system 100 enables the user 102a and the stake-holders 300a-n to set up an account on the web-based platform. For setting the account, the user 102a and the plurality of the stake-holders 300a-n enters a set of information on the web-based platform and/or application. The information includes but may not be limited to personal information. The personal information includes a first name, a last name, an email id, a password, a phone number, an address, a date of birth, a city name, a state name, a country name, academic information, family background and the like. The user 102a and the plurality of the stake-holders 300a-n get registered by entering the personal information on the web-based platform.

Further, the data aggregation system 100 enables the user 102a to enter profile information. The profile information includes the first set of quantitative data and qualitative data. The quantitative data includes input metrics, uploaded audios/videos and the like (described later). The qualitative data includes input ratings, points, comments, messages and the like (described later). In an embodiment of the present disclosure, the user 102a enters sports related information including types of sports played, position in each of the sports played, names of previous and current coaches, names of previous clubs the user 102a has represented, names of current clubs the user 102a is representing, diet plans, practice plans, levels at which the user 102a has played (for example, club level, international tournaments and the like), skills related to the sports played and the like.

In an embodiment of the present disclosure, the user 102a may play and enter data related to more than one sport. It may be noted that the scope of the invention is not limited by entering the above stated information as profile information; those skilled in the art would appreciate that the user 102a may enter other related information as well. In an embodiment of the present disclosure, the guardians 300e may enter the above stated information on behalf of their children (the user 102a) on the web-based platform. In another embodiment, the plurality of coaches 300a may enter the above stated information or other information pertaining to the user 102a on the web-based platform.

In another embodiment, each of the user 102a enters information including coachability, attitude, work ethic, communication skills, listening skills, grasping power of concepts, rapport with coaches and team mates, competitiveness, mental toughness, athleticism, sports sense, on-field exercise routine, off-field exercise routine, calendars/schedules and the like. It may be noted that the scope of the invention is not limited by entering the above stated information such as the profile information; those skilled in the art would appreciate that the user 102a may enter other related information as well. In an embodiment of the present disclosure, the guardians 300e may enter the above stated information on behalf of their children (the user 102a) or any other pertinent information on the web-based platform. In another embodiment, the plurality of coaches 300a may enter the above stated information or any other information of the user 102a or on behalf of the user 102a on the web-based platform.

For example, a user X plays ice hockey at a right winger position and a user Y plays at a center position for an ice hockey team Z coached by a coach Z1. Further, and a user A plays basketball at a left forward position and a user B plays at a right forward position for a basketball club team C coached by a coach C1. The user X, the user Y, the user A and the user B login to the data aggregation system 100. The user X and the user Y enter a name of a sport played (the ice hockey), a name of the ice hockey team A (say New York Rangers), a playing position (the right winger position for the user X and the center position for the user Y), a name of the coach Z1, number of on-field practice hours (say 4 hours for the user X and 6 hours for the user Y), number of off-field practice hours (say 6 hours for the user X and 8 hours for the user Y) and a diet plan (say carbohydrates and protein for the user X and fats for the user Y). The user A and the user B enter a name of a sport played (the basketball), a name of the basketball team C (say LA Lakers), a playing position (the left forward position for the user A and the right forward position for the user B), a name of the coach C1, number of on-field practice hours (say 4 hours for the user A and 6 hours for the user B), number of off-field practice hours (say 3 hours for the user A and 4 hours for the user B) and one or more skills for the user A and the user B (say dribbling skills, passing skills, shooting skills and defensive skills).

In an embodiment of the present disclosure, the plurality of coaches 300a enters the information including a name of a sport coached, a name of current team coached, names of previously coached teams, coaching experience, timeline of coaching corresponding to one or more teams, coaching record for the corresponding one or more teams, sporting achievements, playing experience of the sport coached and the like.

In yet another embodiment, the user 102a may upload match and demonstration images, match audios and videos, demonstration audios and videos, drill schematics, article links, and the like. It may be noted that the scope of the invention is not limited by entering the above stated information as the profile information; those skilled in the art would appreciate that the user 102a may enter other related information as well. For example, the users X and Y upload their videos when they scored a goal and the users A and B upload their videos when they shoot a basket. In an embodiment of the present disclosure, the guardians 300e may upload the match and demonstration images, the match audios and videos, the demonstration audios and videos, the drill schematics, the article links, and the like on behalf of their children (the user 102a) on the web-based platform. In another embodiment, the plurality of coaches 300a may upload the audios and videos on behalf of the user 102a on the web-based platform.

In an embodiment of the present disclosure, the user 102a and the plurality of stake-holders 300a-n may upload the video or image directly from their respective communication devices. In another embodiment of the present disclosure the user 102a and the plurality of stake-holders 300a-n may upload the video or image from a library. The video or image may be pre-uploaded in another website associated with a club of the user 102a and a coach.

In an embodiment of the present disclosure, the users 102a may give ratings and/or points to himself based upon a performance in a match or a couple of matches in a tournament. In another embodiment, the plurality of coaches 300a coaching the users 102a may give the ratings, the points, the comments, the messages to the users 102a based upon his performance in a match or a couple of matches in a tournament. In yet another embodiment, the plurality of stake-holders 300a-n may give the ratings, the points, the comments, the messages to the users 102a based upon his performance in a match or a couple of matches in a tournament. In yet another embodiment, the user 102a may/may not rate a coach of the plurality of coaches 300a based on a performance of the team under his role and guidance. In yet another embodiment, the guardians 300e may provide the comments to their children (the user 102a) regarding their performance in the game.

Further, the guardians 300e may provide feedback regarding each of the plurality of coaches 300a. In an embodiment of the present disclosure, the guardians 300e may give comments to the plurality of coaches 300a. For example, a guardian G1 thinks that under a coach C1, the performance of their child (say, the user 102a) has gone below a suitable mark, and thus rates the coach C1 very low. On the other hand, the same guardian G1 thinks that under a coach C2, the performance of their child (say, the user 102a) has further increased, and thus rates the coach C2 very high. In yet another embodiment, the guardians 300e may file a complaint against any coach of the plurality of coaches 300a. In this case, the plurality of club managers 300b and the plurality of governing bodies 300c can communicate and try to resolve the case.

Further, in an embodiment of the present disclosure, the user 102a and the plurality of stake-holders 300a-n may give the ratings by simply writing one or more values. In another embodiment, the user 102a and the plurality of stake-holders 300a-n may give ratings by choosing from a drop down list (say, a list representing poor, average, good, very good and excellent). In yet another embodiment, the user 102a and the plurality of stake-holders 300a-n may give the ratings by using one or more scales. The one or more scales may feature different skills of the user 102a. In yet another embodiment, the guardians 300e may give comments to the user 102a based on their performances. It may be noted that various rating tools (described above) are provided to rate the user 102a based on his skills; however those skilled in the art would appreciate that more rating tools may be provided to rate the users 102a.

In an embodiment of the present disclosure, the user 102a may have the permissions to view each other's profile. Further, the user 102a may provide the ratings and the comments to each other.

Further, the data aggregation system 100 collects the information and the data described above and stores it in the database 110. Further, the data aggregation system 100 collects and maintains a repository of the collected information in the database 110. In an embodiment of the present disclosure, the user 102a may redeem the points obtained/earned by them. In an embodiment of the present disclosure, the data aggregation system 100 collects the first set of quantitative data and qualitative data from third party sources such as web, third party database, or any other source.

Further, the data aggregation system 100 enables an automatic update of the stored information in the database 110 whenever any additional information is entered by the user 102a and the stake-holders 300a-n. In simpler terms, the database 110 gets updated at regular intervals of time with additional information entered by the user 102a and the stake-holders 300a-n. In an embodiment of the present disclosure, previously stored records are also maintained for future references. In other words, the database 110 is dynamic and updates on periodic basis. For example, the user X has updated his address and phone number on the web-based platform, the same gets updated in the database 110 as well. In another example, the user A uploads a video showing number of baskets shot by him in his last match, or enters a change in the diet plan and the practice plan on the web-based platform. The data aggregation system 100 updates these changes in the database 110 as well.

Further, the data aggregation system 100 analyzes the first set of quantitative data and qualitative data uploaded and/or entered by the user 102a. In an embodiment of the present disclosure, the data aggregation system 100 analyzes the performance of the users 102a in a period of time, say, in a year, last five matches, last half an hour of the game and the like. In another embodiment, the data aggregation system 100 analyzes performance of the user 102a under a specific coach and performance of the user 102a affected due to changes in the diet plan, practice plan and the like.

Continuing with the above stated example, the data aggregation system 100 analyzes that the user X has played better under guidance of his new coach rather than his previous coach. Further, the data aggregation system 100 analyzes that the performance of the user X has improved in last 3 months by changing his diet plan, while the performance of the user Y has degraded in last 3 months by changing his exercise plan.

The data aggregation system 100 aggregates the analyzed information at one place. The analyzed information includes analysis of the first set of quantitative data and qualitative data. Further, the data aggregation system 100 stores the aggregated information in the database 110. The aggregated information includes a collective data that influences the performance of the user 102a. For example, a data (say, a change in diet plans, a change in the exercise plans and the like) representing a positive impact on the performance of the user X is aggregated at a place and a data that leads to a negative impact on the performance of the user X is aggregated at another place. Similarly, a data (say, a change in diet plans, and the like) representing a positive impact on the performance of the user A is aggregated at a place and a data (say, the change in the exercise plans) that leads to a negative impact on the performance of the user A is aggregated at another place.

In an embodiment of the present disclosure, the data aggregation system 100 aggregates performance of the user 102a over a period of time, say a month, a year, last 5 matches played and the like. In another embodiment, the data aggregation system 100 aggregates performance of the user 102a under guidance of a coach and the plurality of coaches 300a.

Further, the data aggregation system 100 generates and/or maintains pictorial representations and reports based on the analyzed and the aggregated information. The analyzed and the aggregated information can be pictorially represented as mountains, spiders, graphs, pie charts and the like. In an embodiment of the present disclosure, the pictorial representation illustrates the performance of the user 102a in a month, year or in last five matches. In another embodiment, the pictorial representation illustrates the performance of the user 102a in a game/sport. In yet another embodiment, the pictorial representation illustrates the performance of the user 102a in one or more games/sports. In yet another embodiment, the pictorial representation illustrates different skills of the user 102a in a match. In yet another embodiment, the pictorial representation illustrates different skills of the user 102a in a couple of matches. Further, the pictorial representations may help the user 102a and the plurality of stake-holders 300a-n to further give the ratings, the comments, the points and the like.

In an embodiment of the present disclosure, the data aggregation system 100 enables displaying of a set of the first set of quantitative data and qualitative data, the analyzed first set of quantitative data and qualitative data, and the aggregated first set of quantitative data and qualitative data to each of the plurality of stake-holders 300a-n. For example, the guardians 300e will be able to view one set of the first set of quantitative data and qualitative data, the analyzed first set of quantitative data and qualitative data and the aggregated first set of quantitative data and qualitative data of the user 102a. The plurality of coaches 300a will be able to view another set of the first set of quantitative data and qualitative data, the analyzed first set of quantitative data and qualitative data and the aggregated first set of quantitative data and qualitative data. In an embodiment of the present disclosure, the user 102a will be able to view a sub-set of the first set of quantitative data and qualitative data, the analyzed first set of quantitative data and qualitative data and the aggregated first set of quantitative data and qualitative data. For example, the user 102a may be able to view the ratings provided by his coach but may not be able to view the comments made by the selection committee.

In an embodiment of the present disclosure, an administrator of the data aggregation system 100 sets policies and permission for accessing the information for the user 102a and the plurality of stake-holders 300a-n. In another embodiment, the data aggregation system 100 enables the user 102a to restrict the displaying and accessing of the set of information, the profile information, the analyzed and the aggregated information to the plurality of stake-holders 300a-n. In yet another embodiment, the data aggregation system 100 enables the user 102a to restrict and/or display set of the first set of quantitative data and qualitative data, the analyzed first set of quantitative data and qualitative data and the aggregated first set of quantitative data and qualitative data to the plurality of stake-holders 300a-n.

In an embodiment of the present disclosure, the data aggregation system 100 enables the user 102a to set a privacy of his account. In an embodiment of the present disclosure, the data aggregation system 100 recommends the user 102a to improve specific skills, say, puck skills, skating skills (for the user X and Y) or to follow a specific practice plan, diet plan and the like. In another embodiment of the present disclosure, the data aggregation system 100 recommends the guardians 300e of the user 102a to change and/or give a specific diet to the corresponding user of the user 102a for their better performance. In yet another embodiment of the present disclosure, the data aggregation system 100 recommends the plurality of coaches 300a to follow a specific plan for the user 102a for their better performance.

In yet another embodiment of the present disclosure, the data aggregation system 100 recommends the plurality of governing bodies 300c and the plurality of club managers 300b to rank the user 102a based on the performance analyzed with the help of the pictorial representations. The recommendations are provided based on the analyzed information, the aggregated information, and the pictorial representations.

More specifically, the data aggregation system 100 distributes the quantitative data, the qualitative data, the visualized reports, the pictorial representations and the like to the user 102a, and the plurality of stake-holders 300a-n. Moreover, the recommendations prove to be useful for the plurality of governing bodies 300c and the plurality of club managers 300b during time of selection of the user 102a for one or more tournaments and leagues.

It may be noted that in FIG. 3A, a user 102a interacts with the data aggregation system 100, however those skilled in the art would appreciate that more number of users may interact with the data aggregation system 100.

FIG. 3B illustrates the data aggregation system 100 for aggregating human health related data, in accordance with another embodiment of the present disclosure. It may be noted that to explain the system elements of FIG. 3B, references will be made to the system elements of the FIG. 1.

As shown in FIG. 3B, the user 102a interacts with the data aggregation system 100 through a web server 104. The user 102a is associated with the communication device 106a. Further, the communication device 106a is associated with the web server 104. In simpler terms, the user 102a communicates with the web server 104 through the communication device 106a.

In addition, a plurality of stake-holders 302a-n is associated with the data aggregation system 100. The plurality of stake-holders 302a-n includes a plurality of doctors 302a, a plurality of nurses 302b, a plurality of chemists 302c, a plurality of health care providers 302d, guardians 302e, a plurality of health insurance agents 302f and other stake-holders 302n. Each of the plurality of stake-holders 302a-n interacts with the data aggregation system 100 with their respective communication devices.

The data aggregation system 100 enables the registration of the user 102a on the web-based platform and/or application. Further, the data aggregation system 100 enables registration of each of the plurality of stake-holders 302a-n on the web-based platform and/or application. In an embodiment of the present disclosure, the data aggregation system 100 enables the user 102a and the stake-holders 302a-n to set up an account on the web-based platform. For setting the account, the user 102a and the plurality of the stake-holders 302a-n enters a set of information on the web-based platform and/or application. The information includes but may not be limited to personal information (described above). The user 102a and the plurality of the stake-holders 302a-n get registered by entering the personal information on the web-based platform.

Further, the data aggregation system 100 enables the user 102a to enter profile information. The profile information includes the first set of quantitative data and qualitative data. The quantitative data includes input metrics, uploaded audios/videos and the like (described later). The qualitative data includes input ratings, points, comments, messages and the like (described later). In an embodiment of the present disclosure, the user 102a enters his health related information. The health related information can be disease related information and daily fitness routine information. The disease related information includes medical history of the user 102a including blood group of the user 102a, any acute/chronic disease the user 102a was suffering from, any acute/chronic disease the user 102a is currently suffering, names of his previous doctors/specialists, names of his current doctors/specialists, medicines recommended previously and currently, diet recommended previously and currently, any specific allergies, names of hospitals and health care providers in which the user 102a was/is taking treatment from, reputation and ratings of the hospitals and health care providers mentioned, any specific medical tests the user 102a has gone through, relief from a specific treatment and the like. It may be noted that the scope of the invention is not limited by entering the above stated information as profile information; those skilled in the art would appreciate that the user 102a may enter other related information as well.

In another embodiment, the user 102a enters his daily fitness routine information. The daily fitness routine information includes his diet plan, exercise plan, sleep hours, work hours, any specific precaution taken to prevent a particular disease and the like. It may be noted that the scope of the invention is not limited by entering the above stated information such as the profile information; those skilled in the art would appreciate that the user 102a may enter other related information as well.

In an embodiment of the present disclosure, the guardians 302e may enter the above stated information on behalf of their children (the user 102a) on the web-based platform. In an embodiment of the present disclosure, the plurality of doctors 302a, the plurality of nurses 302b and the plurality of health insurance agents 302f may enter the above stated information on behalf of user 102a on the web-based platform.

For example, a user X having 50 years of age is suffering from arthritis and a user Y having 23 years of age is suffering from sinus. The user X and the user Y login to the data aggregation system 100. The user X enter his disorder (arthritis), name of his physiotherapist (say, Dr. Todd Kelvin), recommended medicines (say, one or more edible medicines, ointments and the like), recommended exercise (say, yoga), information related to allergies (say, the user X cannot take high doses of medicines), name of health care providers (Manchester diagnostic centre) and the like. Similarly, the user Y enter his disorder (sinus), name of his physiotherapist (say, Dr. James Darwin), recommended medicines (say, avelox oral, cefprozil oral, ampicillin injection and the like), recommended exercise (say, yoga), information related to allergies (say, the user Y cannot take high doses of medicines), name of health care providers (New York rejuvenation point) and the like.

In an embodiment of the present disclosure, the plurality of doctors 302a enters the information including his specialization, a name of a hospital and/or private centre from where he is currently operating, names of his previous hospitals and/or private centers, experience, timeline of working/operating corresponding to one or more hospitals/health care providers, working record, achievements and the like. Further, the plurality of nurses 302b enters the information including a name of a hospital and/or private centre from where she is currently operating, names of previous hospitals and/or private centers, experience, timeline of working/operating corresponding to one or more hospitals/health providers, working record, achievements and the like.

In yet another embodiment, the user 102a may upload reports of various medical test he/she has gone through. It may be noted that the scope of the invention is not limited by entering the above stated information as the profile information; those skilled in the art would appreciate that the user 102a may enter other related information as well. For example, the users X and Y upload their reports when they had gone through ultrasounds of their knee area and nose respectively. In an embodiment of the present disclosure, the guardians 302e may upload the reports of various medical tests the user 102a has gone through on their behalf on the web-based platform. In another embodiment, the plurality of doctors 302a and the plurality of nurses 302b may upload the reports of various medical tests the user 102a has gone through on behalf of the user 102a on the web-based platform.

In an embodiment of the present disclosure, the user 102a and the plurality of stake-holders 302a-n may upload the reports directly from their respective communication devices. In another embodiment of the present disclosure the user 102a and the plurality of stake-holders 302a-n may upload the reports from a library. The reports may be pre-uploaded in another website associated with a health care provider of the user 102a.

In an embodiment of the present disclosure, the users 102a may give ratings and/or points to the plurality of doctors 302a, the plurality of nurses 302b, the plurality of chemists 302c, the plurality of health care providers 302d and the like based upon their respective roles in helping and/or treating the user 102a. In another embodiment, the guardians 302e may give the ratings, the points, the comments, the messages to the plurality of doctors 302a, the plurality of nurses 302b, the plurality of chemists 302c, the plurality of health care providers 302d and the like on behalf of their children (the user 102a) based upon their respective roles in helping and/or treating the user 102a.

Further, the guardians 302e may provide feedback regarding each of the plurality of doctors 302a, the plurality of nurses 302b, the plurality of chemists 302c and the plurality of health care providers 302d. In another embodiment, the user 102a may provide feedback regarding each of the plurality of doctors 302a, the plurality of nurses 302b, the plurality of chemists 302c and the plurality of health care providers 302d. In an embodiment of the present disclosure, the guardians 302e may give comments to the plurality of doctors 302a. For example, a guardian G1 thinks that under a doctor D1 and a health care provider H1, the health of her child (the user 102a) has gone below a suitable mark, and thus rates the doctor D1 and the health care provider H1 very low. On the other hand, the same guardian G1 thinks that under a doctor D2 and a health care provider H2, the performance of her child (the user 102a) has further increased, and thus rates the doctor D2 and the health care provider H2 very high.

Further, in an embodiment of the present disclosure, the user 102a and the plurality of stake-holders 302a-n may give ratings by simply writing one or more values. In another embodiment, the user 102a and the plurality of stake-holders 302a-n may give ratings by choosing from a drop down list (say, a list representing poor, average, good, very good and excellent). In yet another embodiment, the user 102a and the plurality of stake-holders 302a-n may give the ratings by using one or more scales. In yet another embodiment, the guardians 302e may give comments to the user 102a and the plurality of stake-holders 302a-n. It may be noted that various rating tools (described above) are provided to rate the user 102a; however those skilled in the art would appreciate that more rating tools may be provided to rate the users 102a. In an embodiment of the present disclosure, the user 102a may have the permissions to view each other's profile. Further, the plurality of doctors 302a and the plurality of nurses 302b may provide the ratings and the comments to each other.

Further, the data aggregation system 100 collects the information and the data described above and stores it in the database 110. Further, the data aggregation system 100 collects and maintains a repository of the collected information in the database 110. In an embodiment of the present disclosure, the data aggregation system 100 collects the first set of quantitative data and qualitative data from third party sources such as web, third party database, or any other source.

Further, the data aggregation system 100 enables an automatic updation of the stored information in the database 110 whenever any additional information is entered by the user 102a and the stake-holders 302a-n. In simpler terms, the database 110 gets updated at regular intervals of time with the additional information entered by the user 102a and the stake-holders 302a-n. In an embodiment of the present disclosure, previously stored records are also maintained for future references. In other words, the database 110 is dynamic and updates on periodic basis. For example, the user X has updated his address and phone number on the web-based platform, the same gets updated in the database 110 as well. In another example, the user Y uploads a report of his medical test on the web-based platform. The data aggregation system 100 updates these changes in the database 110 as well.

Further, the data aggregation system 100 analyzes the first set of quantitative data and qualitative data uploaded and/or entered by the user 102a. In an embodiment of the present disclosure, the data aggregation system 100 analyzes the health of the user 102a in a period of time, say, in a year, and the like. In another embodiment, the data aggregation system 100 analyzes health of the user 102a under a specific doctor and health of the user 102a affected due to changes in the diet plan, medicines and the like.

Continuing with the above stated example, the data aggregation system 100 analyzes that the medical condition of the user X has improved under his new doctor rather than his previous doctor. Further, the data aggregation system 100 analyzes that the medical condition of the user X has improved in last 3 months by changing his diet plan and the medicines, while the medical condition of the user Y has degraded in last 3 months by changing his diet plan and the medicines.

The data aggregation system 100 aggregates the analyzed information at one place. The analyzed information includes analysis of the first set of quantitative data and qualitative data. Further, the data aggregation system 100 stores the aggregated information in the database 110. The aggregated information includes a collective data that influences the health of the user 102a. For example, a data (say, a change in diet plans, a change in the exercise plans, a change in medicines, a change in doctors and the like) representing a positive impact on the medical condition of the user X is aggregated at a place and a data that leads to a negative impact on the medical condition of the user X is aggregated at another place. Similarly, a data (say, a change in diet plans, a change in medicines, a change in doctors and the like) representing a positive impact on the medical condition of the user Y is aggregated at a place and a data (say, the change in the exercise plans) that leads to a negative impact on the medical condition of the user Y is aggregated at another place.

In an embodiment of the present disclosure, the data aggregation system 100 aggregates the medical condition of the user 102a over a period of time, say a month, a year, last 5 years and the like. In another embodiment, the data aggregation system 100 aggregates the medical condition of the user 102a under supervision of a doctor and the plurality of doctors 302a.

Further, the data aggregation system 100 generates and/or maintains pictorial representations and the reports based on the analyzed and the aggregated information. The analyzed and the aggregated information can be pictorially represented as the mountains, the spiders, the graphs, the pie charts and the like. In an embodiment of the present disclosure, the pictorial representation illustrates the medical condition of the user 102a in a month, year or in last five years. In another embodiment, the pictorial representation illustrates the medical condition of the user 102a in a medical disorder. In yet another embodiment, the pictorial representation illustrates the medical condition of the user 102a in one or more medical disorders.

The pictorial representations may help the plurality of doctors 302a, the plurality of nurses 302b, the plurality of chemists 302c and the plurality of health care providers 302d to follow a better and a specific treatment for the user 102a. Further, the pictorial representations may help the user 102a to further give the ratings, the comments, the points and the like to the plurality of doctors 302a, the plurality of nurses 302b, the plurality of chemists 302c and the plurality of health care providers 302d. Furthermore, the pictorial representations may help the guardians 302e to further give the ratings, the comments, the points and the like to the plurality of doctors 302a, the plurality of nurses 302b, the plurality of chemists 302c and the plurality of health care providers 302d. In an embodiment of the present disclosure, the plurality of health insurance agents 302f may view the pictorial representations and contact and/or help the user 102a to attain his/her medical insurance.

In an embodiment of the present disclosure, the data aggregation system 100 enables displaying of a set of the first set of quantitative data and qualitative data, the analyzed first set of quantitative data and qualitative data and the aggregated first set of quantitative data and qualitative data to each of the plurality of stake-holders 302a-n. For example, the guardians 302e will be able to view one set of the first set of quantitative data and qualitative data, the analyzed first set of quantitative data and qualitative data and the aggregated first set of quantitative data and qualitative data of the user 102a. The plurality of doctors 302a will be able to view another set of the first set of quantitative data and qualitative data, the analyzed first set of quantitative data and qualitative data and the aggregated first set of quantitative data and qualitative data.

In an embodiment of the present disclosure, the administrator of the data aggregation system 100 sets the policies and permission for accessing the information for the users 102a and the plurality of stake-holders 302a-n. In another embodiment, the data aggregation system 100 enables the user 102a to restrict displaying and accessing of the set of information, the profile information, the analyzed and the aggregated information to the plurality of stake-holders 302a-n. In yet another embodiment, the data aggregation system 100 enables the user 102a to restrict and/or display set of the first set of quantitative data and qualitative data, the analyzed first set of quantitative data and qualitative data and the aggregated first set of quantitative data and qualitative data to the plurality of stake-holders 302a-n.

In an embodiment of the present disclosure, the data aggregation system 100 enables the user 102a to set a privacy of his account. In an embodiment of the present disclosure, the data aggregation system 100 recommends the user 102a to follow a specific diet plan, and/or change his doctor or the health care provider. In another embodiment of the present disclosure, the data aggregation system 100 recommends the guardians 302e of the user 102a to change and/or give a specific diet to the user 102a for his better recovery. In yet another embodiment of the present disclosure, the data aggregation system 100 recommends the plurality of doctors 302a and the plurality of nurses 302b to follow a specific treatment plan for the user 102a for improving their health. In yet another embodiment of the present disclosure, the data aggregation system 100 recommends the plurality of doctors 302a and the plurality of nurses 302b not to give a specific type of medicine in case the user 102a has one or more allergies.

The recommendations are provided based on the analyzed information, the aggregated information, and the pictorial representations. More specifically, the data aggregation system 100 distributes the quantitative data, the qualitative data, the visualized reports, the pictorial representations and the like to the user 102a, and the plurality of stake-holders 302a-n. Moreover, the recommendations prove to be useful for the plurality of health care providers 302d during time of selection/recruitment of the plurality of doctors 302a and the plurality of nurses 302b.

It may be noted that in FIG. 3B, a user 102a interacts with the data aggregation system 100, however those skilled in the art would appreciate that more number of users may interact with the data aggregation system 100.

FIG. 3C illustrates the data aggregation system 100 for aggregating human education related data, in accordance with yet another embodiment of the present disclosure. It may be noted that to explain the system elements of FIG. 3C, references will be made to the system elements of the FIG. 1.

As shown in FIG. 3C, the user 102a interacts with the data aggregation system 100 through a web server 104. The user 102a is associated with the communication device 106a. Further, the communication device 106a is associated with the web server 104. In simpler terms, the user 102a communicates with the web server 104 through the communication device 106a.

In addition, a plurality of stake-holders 304a-n is associated with the data aggregation system 100. The plurality of stake-holders 304a-n includes a plurality of teachers 304a, a plurality of private tutors 304b, guardians 304c, a plurality of governing bodies 304d, a plurality of supervisors 304e and other stake-holders 304n. Each of the plurality of stake-holders 304a-n interacts with the data aggregation system 100 with their respective communication devices.

The data aggregation system 100 enables the registration of the user 102a on the web-based platform and/or application. Further, the data aggregation system 100 enables registration of each of the plurality of stake-holders 304a-n on the web-based platform and/or application. In an embodiment of the present disclosure, the data aggregation system 100 enables the user 102a and the stake-holders 304a-n to set up an account on the web-based platform. For setting the account, the user 102a and the plurality of the stake-holders 304a-n enters a set of information on the web-based platform and/or application. The information includes but may not be limited to personal information (described above). The user 102a and the plurality of the stake-holders 304a-n get registered by entering the personal information on the web-based platform.

Further, the data aggregation system 100 enables the user 102a to enter a profile information. The profile information includes the first set of quantitative data and qualitative data. The quantitative data includes input metrics, uploaded audios/videos of extra-curricular activities performed by the user 102a and the like (described later). The qualitative data includes input ratings, points, comments, messages and the like (described later).

In an embodiment of the present disclosure, the user 102a enters an academic and a non-academic information. The academic information includes current standard/year of the user 102a, grades in each subject in a first set of exams (say, mid-term exams, first semester exams and the like), grades in each subject in a second set of exams (say, final-term exams, second semester exams and the like), an overall grade of the user 102a in each subject over a period of years, an overall grade of the user 102a over a period of years, favorite subjects, names of institutions and universities under which the user 102a has studied or currently studying, names of subject teachers in each standard/year, number of subjects studied in each standard/year and the like. It may be noted that the scope of the invention is not limited to entering the above stated information as profile information; those skilled in the art would appreciate that each of the user 102a may enter other related information as well.

The non-academic information includes data related to non-academic activities or the extra-curricular activities the user 102a has participated in. The extra-curricular activities include but may not be limited to sports, music and dance, and dramatics. Further, the non-academic information includes number of the extra-curricular activities performed in the each standard/year, favorite extra-curricular activity, information about inter and intra institution/universities competitions, and the like. In an embodiment of the present disclosure, the user 102a may enter data related to more than one extra-curricular activity. It may be noted that the scope of the invention is not limited to entering the above stated information as profile information; those skilled in the art would appreciate that each of the user 102a may enter other related information as well. In an embodiment of the present disclosure, the guardians 304c may enter the above stated information on behalf of their children (the user 102a) on the web-based platform. In another embodiment, the plurality of teachers 304a may enter the above stated information or other information pertaining to the user 102a on the web-based platform.

For example, a user X1 studies in a school S1. The user X1 enters his grades obtained in each of the standards, say, A grade in sixth standard, B grade in seventh standard, A grade in eighth standard and so on. Further, the user X1 enters mathematics as his favorite subject. Furthermore, the user X1 is good at dramatics and enters the grades obtained in the dramatics in one or more years. Similarly, another user X2 studies in a school S2. The user X2 enters his grades obtained in each of the standards, say, B grade in sixth standard, C grade in seventh standard, B grade in eighth standard and so on. Further, the user X2 enters English as his favorite subject. Furthermore, the user X2 is good at gymnastics and enters the grades obtained in the gymnastics in one or more years.

In another embodiment, the user 102a enters information including attitude, communication skills, listening skills, grasping power of concepts, rapport with teachers and class mates, competitiveness, mental toughness, calendars/schedules and the like. It may be noted that the scope of the invention should not be limited to enter the profile information; those skilled in the art would appreciate that each of the user 102a may enter other related information as well. In an embodiment of the present disclosure, the guardians 304c may enter the above stated information on behalf of their children (the user 102a) or any other pertinent information on the web-based platform. In another embodiment, the plurality of teachers 304a may enter the above stated information or any other information of the user 102a on their behalf on the web-based platform.

For example, the user X1 enters a list of his upcoming exams, information related to time devoted for studies, say, a total of 8 hours daily, time devoted for improving the communication skills and the listening skills, say, a total of 1 hour daily, time devoted for improving performance in the dramatics and the like. Similarly, the user X2 enters a list of his upcoming exams, information related to time devoted for studies, say, a total of 6 hours daily, time devoted for improving the communication skills and the listening skills, say, a total of half an hour daily, time devoted for improving performance in the gymnastics and the like.

In an embodiment of the present disclosure, the plurality of teachers 304a and the plurality of private tutors 304b enters his own information on the web-based platform including their subject specialization, experience of teaching, names of the institutions/universities in which the plurality of teachers 304a and the plurality of private tutors 304b has taught previously, names of the institutions/universities in which the plurality of teachers 304a and the plurality of private tutors 304b is currently teaching, style of teaching, results achieved by various students under him/her and the like. In yet another embodiment, the user 102a may upload audios and videos of their performance in the extra-curricular activities. For example, the user X1 and X2 upload their videos of a drama competition and a gymnastics competition respectively. In an embodiment of the present disclosure, the guardians 304c may upload the audios and videos of the performance of their children (the user 102a) in the extra-curricular activities on behalf of their children (the user 102a) on the web-based platform. In another embodiment, the plurality of teachers 304a may upload the audios and videos of the performance of the user 102a in the extra-curricular activities on behalf of the user 102a on the web-based platform.

In an embodiment of the present disclosure, the user 102a may enter their academic and non-academic achievements. In an embodiment of the present disclosure, the user 102a may enter number of awards, certificates, medals and the like achieved by them, both in the academic and the non-academic arenas. In another embodiment of the present disclosure, the user 102a give ratings and/or points to themselves based upon their performance in the academic and the non-academic arenas (for example, in a mid-term exam, in a final term exam, in the drama competition and the like). In yet another embodiment, the plurality of teachers 304a teaching the user 102a may give the ratings, the points, the comments, the messages to each of the user 102a based upon their performance in the academic and the non-academic arenas (for example, in the mid-term exam, in the final term exam, in the drama competition and the like). In yet another embodiment, the plurality of stake-holders 304a-n may give the ratings, the points, the comments, the messages to the user 102a based upon their performance in the academic and the non-academic arenas. In yet another embodiment, the user 102a may rate a teacher of the plurality of teachers 304a based on his performance under his/her role and guidance. In yet another embodiment, the guardians 304c may provide the comments to their children (the user 102a) regarding their performance in the in the academic and the non-academic arenas.

Further, the guardians 304c may provide feedback regarding each of the plurality of teachers 304a. In an embodiment of the present disclosure, the guardians 304c may give comments to the plurality of teachers 304a. In another embodiment of the present disclosure, the guardians 304c may give comments to the plurality of private tutors 304b. For example, a guardian G1 thinks that under a private tutor T1, the performance of their child (say, the user 102a) has gone below a suitable mark, and thus rates the private tutor T1 very low. On the other hand, the same guardian G1 thinks that under a private tutor T2, the performance of their child (say, the user 102a) has further increased, and thus rates the private tutor T2 very high. In yet another embodiment, the guardians 304c may file a complaint against any teacher of the plurality of teachers 304a. In this case, the plurality of governing bodies 304d and the plurality of supervisors 304e can communicate and try to resolve the case.

Further, in an embodiment of the present disclosure, the user 102a and the plurality of stake-holders 304a-n may give the ratings by simply writing one or more values. In another embodiment, the user 102a and the plurality of stake-holders 304a-n may give ratings by choosing from a drop down list (say, a list representing poor, average, good, very good and excellent). In yet another embodiment, the user 102a and the plurality of stake-holders 304a-n may give the ratings by using one or more scales. In yet another embodiment, the guardians 304c may give comments to the user 102a based on the performances of the user 102a. It may be noted that various rating tools (described above) are provided to rate the user 102a based on their skills; however, those skilled in the art would appreciate that more rating tools may be provided to rate the user 102a.

In an embodiment of the present disclosure, the user 102a may have permissions to view each other's profile. Further, the user 102a may provide the ratings and the comments to each other.

Further, the data aggregation system 100 collects the information and the data described above and stores it in the database 110. Further, the data aggregation system 100 collects and maintains a repository of the collected information in the database 110. In an embodiment of the present disclosure, the user 102a may redeem the points obtained/earned by them. For example, the points obtained/earned by each of the user 102a may be redeemed for obtaining fee concessions. In an embodiment of the present disclosure, the data aggregation system 100 collects the first set of quantitative data and qualitative data from third party sources such as web, third party database, or any other source.

Further, the data aggregation system 100 enables an automatic update of the stored information in the database 110 whenever any additional information is entered by the user 102a and the stake-holders 304a-n. In simpler terms, the database 110 gets updated at regular intervals of time with the additional information entered by the user 102a and the stake-holders 304a-n. In an embodiment of the present disclosure, previously stored records are also maintained for future references. In other words, the database 110 is dynamic and updated on periodic basis.

For example, the user X1 has updated his house address and name of his school on the web-based platform, the same gets updated in the database 110 as well. In another example, the user X2 uploads a video showing his performance in the gymnastics tournament, or enters a change in the diet plan on the web-based platform. The data aggregation system 100 updates these changes in the database 110 as well.

The data aggregation system 100 analyzes the first set of quantitative data and qualitative data uploaded and/or entered by the user 102a. In an embodiment of the present disclosure, the data aggregation system 100 analyzes performance of the user 102a in both the academic and the non-academic arenas in a period of time, say, in a year, last five months, last five years and the like. In another embodiment, the data aggregation system 100 analyzes performance of the user 102a under a specific teacher/mentor and performance of the user 102a affected due to changes in the study plan, playing hours, sleep hours and the like.

Continuing with the above stated example, the data aggregation system 100 analyzes that the user X1 has scored better grades under guidance of his new teacher ‘A’ rather than his previous teacher ‘B’. Further, the data aggregation system 100 analyzes that user X1 has scored better grades under guidance of his new private tutor ‘T’ rather than his previous private tutor ‘Y’. Furthermore, the data aggregation system 100 analyzes that the performance of the user X1 in the dramatics has improved in last 3 months by changing his practice plan, while the performance of the user X2 in the gymnastics has degraded in last few months by changing his exercise plan. In addition, the data aggregation system 100 analyzes that user X2 has scored slightly poor grades under guidance of his new teacher ‘A1’ rather than his previous teacher ‘B1’. Further, the data aggregation system 100 analyzes that user X2 has scored better grades in English under guidance of his new English teacher rather than his previous English teacher.

The data aggregation system 100 aggregates the analyzed information at one place. The analyzed information includes analysis of the first set of quantitative data and qualitative data. Further, the data aggregation system 100 stores the aggregated information in the database 110. The aggregated information includes a collective data that influences the performance of the user 102a both in the academic and the non-academic arenas. For example, a data (say, a change in self-study hours, a change in the sleep hours and the like) representing a positive impact on the performance of the user X1 in his studies is aggregated at a place and a data that leads to a negative impact on the performance of the user X1 in his studies is aggregated at another place. Similarly, a data (say, a change in self-study hours, a change in the sleep hours and the like) representing a positive impact on the performance of the user X2 in his studies is aggregated at a place and a data that leads to a negative impact on the performance of the user X2 in his studies is aggregated at another place.

In an embodiment of the present disclosure, the data aggregation system 100 aggregates performance of the user 102a over a period of time, say a month, a year, last 5 years and the like. In another embodiment, the data aggregation system 100 aggregates performance of the user 102a under guidance of the teacher/mentor and the plurality of teachers 304a.

Further, the data aggregation system 100 generates and/or maintains pictorial representations and reports based on the analyzed and the aggregated information. The analyzed and the aggregated information can be pictorially represented as the mountains, the spiders, the graphs, the pie charts and the like. In an embodiment of the present disclosure, the pictorial representation illustrates the academic performance of the user 102a in a month, year or in last five years. In another embodiment, the pictorial representation illustrates the non-academic performance of the user 102a in the month, year or in last five years. In yet another embodiment, the pictorial representation illustrates the performance of the user 102a in a subject. In yet another embodiment, the pictorial representation illustrates the performance of the user 102a in one or more subjects. In yet another embodiment, the pictorial representation illustrates different skills of the user 102a in an extra-curricular activity. In yet another embodiment, the pictorial representation illustrates different skills of the user 102a in one or more extra-curricular activities. Further, the pictorial representations may help the user 102a and the plurality of stake-holders 304a-n to further give the ratings, the comments, the points and the like.

In an embodiment of the present disclosure, the data aggregation system 100 enables displaying of a set of the first set of quantitative data and qualitative data, the analyzed first set of quantitative data and qualitative data and the aggregated first set of quantitative data and qualitative data to each of the plurality of stake-holders 304a-n. For example, the guardians 304c will be able to view one set of the first set of quantitative data and qualitative data, the analyzed first set of quantitative data and qualitative data and the aggregated first set of quantitative data and qualitative data of the user 102a. The plurality of teachers 304a will be able to view another set of first set of quantitative data and qualitative data, the analyzed first set of quantitative data and qualitative data and the aggregated first set of quantitative data and qualitative data. In an embodiment of the present disclosure, the user 102a will be able to view a sub-set of the first set of quantitative data and qualitative data, the analyzed first set of quantitative data and qualitative data and the aggregated first set of quantitative data and qualitative data. For example, the user 102a may be able to view the ratings provided by his teacher but may not be able to view the comments made by the plurality of governing bodies 304d.

In an embodiment of the present disclosure, the administrator of the data aggregation system 100 sets the policies and permission for accessing the information for the user 102a and the plurality of stake-holders 304a-n. In another embodiment, the data aggregation system 100 enables the user 102a to restrict displaying and accessing of the set of information, the profile information, the analyzed and the aggregated information to the plurality of stake-holders 304a-n. In yet another embodiment, the data aggregation system 100 enables the user 102a to restrict and/or display the set of the first set of quantitative data and qualitative data, the analyzed first set of quantitative data and qualitative data and the aggregated first set of quantitative data and qualitative data to the plurality of stake-holders 304a-n.

In an embodiment of the present disclosure, the data aggregation system 100 enables the user 102a to set a privacy of his account. In an embodiment of the present disclosure, the data aggregation system 100 recommends the user 102a to make changes in the number of sleep hours and the self-study hours or to follow a specific time table to manage both academic and non-academic activities simultaneously. In another embodiment of the present disclosure, the data aggregation system 100 recommends the guardians 304c of the user 102a to change and/or give a specific diet to the user 102a for their better performance in both the academic and non-academic activities. In yet another embodiment of the present disclosure, the data aggregation system 100 recommends the plurality of teachers 304a to follow a specific style of teaching for better performance of the user 102a.

In yet another embodiment of the present disclosure, the data aggregation system 100 recommends the plurality of governing bodies 304d and the plurality of supervisors 304e to rank the user 102a based on the performance analyzed with the help of the pictorial representations. The recommendations are provided based on the analyzed information, the aggregated information, and the pictorial representations. More specifically, the data aggregation system 100 distributes the quantitative data, the qualitative data, the visualized reports, the pictorial representations and the like to the user 102a, and the plurality of stake-holders 304a-n. Moreover, in an embodiment, the recommendations prove to be useful for the plurality of governing bodies 304d and the plurality of supervisors 304e while deciding a scholarship holder. Further, in another embodiment, the recommendations prove to be useful for the plurality of governing bodies 304d and the plurality of supervisors 304e while deciding mentors for the user 102a. For example, if the user X1 is weak in algebra and the pictorial representations suggest that a teacher Y has an excellent past record of improving grades in the algebra, then the data aggregation system 100 may recommend the plurality of supervisors 304e to provide the user X1 a mentor (the teacher Y) for improving his grades in the algebra.

It may be noted that in FIG. 3C, a user 102a interacts with the data aggregation system 100, however those skilled in the art would appreciate that more number of users may interact with the data aggregation system 100.

FIG. 3D illustrates the data aggregation system 100 for aggregating human music related data, in accordance with yet another embodiment of the present disclosure. It may be noted that to explain the system elements of FIG. 3D, references will be made to the system elements of the FIG. 1.

As shown in FIG. 3D, the user 102a interacts with the data aggregation system 100 through a web server 104. The user 102a is associated with the communication device 106a. Further, the communication device 106a is associated with the web server 104. In simpler terms, the user 102a communicates with the web server 104 through the communication device 106a.

In addition, a plurality of stake-holders 306a-n is associated with the data aggregation system 100. The plurality of stake-holders 306a-n includes a plurality of music teachers 306a, a plurality of talent agencies 306b, guardians 306c, a plurality of music institutes 306d and other stake-holders 306n. Each of the plurality of stake-holders 306a-n interacts with the data aggregation system 100 with their respective communication devices.

The data aggregation system 100 enables the registration of the user 102a on the web-based platform and/or application. Further, the data aggregation system 100 enables registration of each of the plurality of stake-holders 306a-n on the web-based platform and/or application. In an embodiment of the present disclosure, the data aggregation system 100 enables the user 102a and the stake-holders 306a-n to set up an account on the web-based platform. For setting the account, the user 102a and the plurality of the stake-holders 306a-n enters a set of information on the web-based platform and/or application. The information includes but may not be limited to personal information (described above). The user 102a and the plurality of the stake-holders 306a-n get registered by entering the personal information on the web-based platform.

Further, the data aggregation system 100 enables the user 102a to enter a profile information. The profile information includes the first set of quantitative data and qualitative data. The quantitative data includes input metrics, uploaded audios/videos of music events/concerts and the like (described later). The qualitative data includes input ratings, points, comments, messages and the like (described later).

In an embodiment of the present disclosure, the user 102a enters information including his/her music skills (for example, instruments played by the user 102a, singing, types of dances performed, music composing, lyrics and the like), various levels of competitions in which the user 102a has performed, achievements, names of previous and current music teachers, names of previous and current music institutes, diet plan, exercise plan, associations with a band, name of the bands with which the user 102a has performed and the like. In an embodiment of the present disclosure, the user 102a may enter data related to more than one music skills. It may be noted that the scope of the invention is not limited to entering the above stated information as profile information; those skilled in the art would appreciate that each of the user 102a may enter other related information as well.

For example, a user X is a rock singer and enters his music skills (rock singing, guitar playing and the like), levels performed (national and international), achievements (5 international awards and 10 national awards), name of current teacher (Alex Brown), name of his band (Mystic group) and the like. Similarly, a user Y is a dancer and enters his dancing skills (hip-hop, lyrical hip-hop, jazz and the like), levels performed (national and international), achievements (3 international awards and 15 national awards), name of current teacher (Christine Velvet) and the like.

In an embodiment of the present disclosure, the guardians 306c may enter the above stated information on behalf of their children or any other pertinent information (the user 102a) on the web-based platform. In another embodiment, the plurality of music teachers 306a may enter the above stated information or any other information of the user 102a on behalf of the user 102a on the web-based platform.

For example, the user X enters a list of his upcoming events and competitions, information related to time devoted for practice, say, a total of 6 hours daily and the like. Similarly, the user Y enters a list of his upcoming events and competitions, information related to time devoted for practice, say, a total of 7 hours daily and the like.

In an embodiment of the present disclosure, the plurality of music teachers 306a may enter their own information on the web-based platform including specialization, experience in teaching, names of the institutes they have worked with, name of the institutes/group they are currently working, performance of various users under their respective guidance and the like.

In yet another embodiment, the user 102a may upload audios and videos of their performances. For example, the users X and Y upload their videos of a music competition and a dancing competition respectively. In an embodiment of the present disclosure, the guardians 306c may upload the audios and videos of the performance of their children (the user 102a) on their behalf on the web-based platform. In another embodiment, the plurality of music teachers 306a may upload the audios and videos of the performance of the user 102a on their behalf on the web-based platform.

In an embodiment of the present disclosure, the user 102a gives ratings and/or points to himself based upon their performance in the music and/or dance competitions. In another embodiment, the plurality of teachers 306a may give the ratings, the points, comments, the messages to the user 102a based upon their performance. In yet another embodiment, the plurality of stake-holders 306a-n may give the ratings, the points, the comments, the messages to the user 102a based upon their performance in the competitions and/or events. In yet another embodiment, the user 102a may rate a teacher of the plurality of music teachers 306a based on their performance under his/her role and guidance. In yet another embodiment, the guardians 306c may provide the comments to their children (the user 102a) regarding their performance in the competitions and/or events. In yet another embodiment of the present disclosure, the plurality of music teachers 306a gives ratings and/or points to themselves based upon performance of their students in the music and/or dance competitions.

Further, the guardians 306c may provide feedback regarding each of the plurality of music teachers 306a. In an embodiment of the present disclosure, the guardians 306c may give comments to the plurality of music teachers 306a. Further, in an embodiment of the present disclosure, the user 102a and the plurality of stake-holders 306a-n may give the ratings by simply writing one or more values. In another embodiment, the user 102a and the plurality of stake-holders 306a-n may give ratings by choosing from a drop down list (say, a list representing poor, average, good, very good and excellent). In yet another embodiment, the user 102a and the plurality of stake-holders 306a-n may give the ratings by using one or more scales. In yet another embodiment, the guardians 306c may give comments to the user 102a based on their performances. It may be noted that various rating tools (described above) are provided to rate the user 102a based on their skills; however, those skilled in the art would appreciate that more rating tools may be provided to rate the user 102a. In an embodiment of the present disclosure, the user 102a may have permissions to view other user's profile. Further, the user 102a may provide the ratings and the comments to each other.

Further, the data aggregation system 100 collects the information and the data described above and stores it in the database 110. Further, the data aggregation system 100 collects and maintains a repository of the collected information in the database 110. In an embodiment of the present disclosure, the user 102a may redeem the points obtained/earned by them. In an embodiment of the present disclosure, the data aggregation system 100 collects the first set of quantitative data and qualitative data from third party sources such as web, third party database, or any other source.

Further, the data aggregation system 100 enables an automatic update of the stored information in the database 110 whenever any additional information is entered by the user 102a and the stake-holders 306a-n. In simpler terms, the database 110 gets updated at regular intervals of time with the information entered by the user 102a and the stake-holders 306a-n. In an embodiment of the present disclosure, previously stored records are also maintained for future references. In other words, the database 110 is dynamic and updated on periodic basis.

For example, the user X has added playing drums as his new skill on the web-based platform; the same gets updated in the database 110 as well. In another example, the user Y uploads a video showing his performance in a dance show on the web-based platform. The data aggregation system 100 updates these changes in the database 110 as well.

The data aggregation system 100 analyzes the first set of quantitative data and qualitative data uploaded and/or entered by the user 102a. In an embodiment of the present disclosure, the data aggregation system 100 analyzes performance of the user 102a in a period of time, say, in a year, last five months, last five years and the like. In another embodiment, the data aggregation system 100 analyzes performance of the user 102a under a specific music teacher/mentor.

The data aggregation system 100 aggregates the analyzed information at one place. The analyzed information includes analysis of the first set of quantitative data and qualitative data. Further, the data aggregation system 100 stores the aggregated information in the database 110. The aggregated information includes a collective data that influences the performance of the user 102a. For example, a data (say, a change in practice hours and the like) representing a positive impact on the performance of the user X is aggregated at a place and a data that leads to a negative impact on the performance of the user X is aggregated at another place. Similarly, a data (say, a change in dancing style, expressions, practice hours and the like) representing a positive impact on the performance of the user Y is aggregated at a place and a data that leads to a negative impact on the performance of the user Y is aggregated at another place.

In an embodiment of the present disclosure, the data aggregation system 100 aggregates performance of the user 102a over a period of time, say a month, a year, last 5 years and the like. In another embodiment, the data aggregation system 100 aggregates performance of the user 102a under guidance of the music teacher.

Further, the data aggregation system 100 generates and/or maintains pictorial representations and reports based on the analyzed and the aggregated information. The analyzed and the aggregated information can be pictorially represented as the mountains, the spiders, the graphs, the pie charts and the like. In an embodiment of the present disclosure, the pictorial representation illustrates the performance of the user 102a in a month, year or in last five years. In yet another embodiment, the pictorial representation illustrates the performance of the user 102a in a skill. In yet another embodiment, the pictorial representation illustrates the performance of the user 102a in one or more skills. Further, the pictorial representations may help the user 102a and the plurality of stake-holders 306a-n to further give the ratings, the comments, the points and the like.

In an embodiment of the present disclosure, the data aggregation system 100 enables displaying of a set of the first set of quantitative data and qualitative data, the analyzed quantitative and qualitative information, and the aggregated quantitative and qualitative information to each of the plurality of stake-holders 306a-n. For example, the guardians 306c will be able to view one set of the first set of quantitative data and qualitative data, the analyzed quantitative and qualitative information, and the aggregated quantitative and qualitative information of the user 102a. The plurality of music teachers 306a will be able to view another set of the first set of quantitative data and qualitative data, the analyzed quantitative and qualitative information, and the aggregated quantitative and qualitative information. In an embodiment of the present disclosure, the user 102a will be able to view a sub-set of the first set of quantitative data and qualitative data, the analyzed quantitative and qualitative information, and the aggregated quantitative and qualitative information. For example, the user 102a may be able to view the ratings provided by his music teacher and other stake-holders 306a-n but may not be able to view the comments made by the plurality of talent agencies 306b.

In an embodiment of the present disclosure, the administrator of the data aggregation system 100 sets the policies and permission for accessing the information for the user 102a and the plurality of stake-holders 306a-n. In another embodiment, the data aggregation system 100 enables the user 102a to restrict displaying and accessing of the set of information, the profile information, the analyzed and the aggregated information to the plurality of stake-holders 306a-n. In yet another embodiment, the data aggregation system 100 enables the user 102a to restrict and/or display the set of the first set of quantitative data and qualitative data, the analyzed quantitative and qualitative information, and the aggregated quantitative and qualitative information to the plurality of stake-holders 306a-n.

In an embodiment of the present disclosure, the data aggregation system 100 enables the user 102a to set a privacy of his account. In an embodiment of the present disclosure, the data aggregation system 100 recommends the user 102a to make changes in the number of practice hours or to follow a specific time table to manage practice of different activities simultaneously. In another embodiment of the present disclosure, the data aggregation system 100 recommends the plurality of music teachers 306a to follow a specific style of training for better performance of the user 102a.

In yet another embodiment of the present disclosure, the data aggregation system 100 recommends the plurality of talent agencies 306b to rank the user 102a based on the performance analyzed with the help of the pictorial representations and further provide better opportunities to the user 102a based on his rankings. The recommendations are provided based on the analyzed information, the aggregated information, and the pictorial representations. More specifically, the data aggregation system 100 distributes the quantitative data, the qualitative data, the visualized reports, the pictorial representations and the like to the user 102a, and the plurality of stake-holders 306a-n. For example, if the user X has received high rankings/ratings by the plurality of stake-holders 306a-n, then the data aggregation system 100 may recommend the plurality of talent agencies 306b to provide better opportunities (say, a singing offer) to the user X.

FIG. 3E illustrates the data aggregation system 100 for aggregating miscellaneous data corresponding to users, in accordance with yet another embodiment of the present disclosure. It may be noted that to explain the system elements of FIG. 3E, references will be made to the system elements of the FIG. 1.

As shown in FIG. 3E, the user 102a interacts with the data aggregation system 100 through a web server 104. The user 102a is associated with the communication device 106a. Further, the communication device 106a is associated with the web server 104. In simpler terms, the user 102a communicates with the web server 104 through the communication device 106a.

In addition, a plurality of stake-holders 308 is associated with the data aggregation system 100. The plurality of stake-holders 308 includes one or more individuals associated with the user 102a (described later). Further, each of the plurality of stake-holders 308 interacts with the data aggregation system 100 with their respective communication devices.

The data aggregation system 100 enables the registration of the user 102a on the web-based platform and/or application (described above). Further, the data aggregation system 100 enables registration of each of the plurality of stake-holders 308 on the web-based platform and/or application. In an embodiment of the present disclosure, the data aggregation system 100 enables the user 102a and the stake-holders 308a to set up an account on the web-based platform. For setting the account, the user 102a and the plurality of the stake-holders 308 enters a set of information on the web-based platform and/or application. The information includes but may not be limited to personal information (described above). The user 102a and the plurality of the stake-holders 308 get registered by entering the personal information on the web-based platform.

Further, the data aggregation system 100 enables the user 102a to enter a profile information. The profile information includes the first set of quantitative data and qualitative data. The quantitative data includes input metrics, uploaded audios/videos and the like (described later). The qualitative data includes input ratings, points, comments, messages and the like (described later).

In an embodiment of the present disclosure, the user 102a enters information including his car, pets, hobbies, house type, shopping preferences (for example, clothing preferences, footwear preferences, color preferences, brands liked by him and the like), travel experiences, movies watched, types of entertainment he likes/prefers, job profile, name of company for which he works, working experience and the like. It may be noted that the scope of the invention is not limited to entering the above stated information as profile information; those skilled in the art would appreciate that each of the user 102a may enter other related information as well. In an embodiment, the plurality of stake-holders 308 may include but not be limited to car retailers, owners of one or more shops of a shopping mall, one or more shopping websites, travel agents, co-workers in office, guardians and friends.

For example, a user X enters information about his car names (for example, Audi R8, and Mercedes Benz CLA class), pets (two dogs), shopping preferences (say, he likes to wear Nike products), travel experience (moves out of his town in every two months), job related information (say, he is a software engineer having an experience of 15 years and works for Google) and the like. Similarly, a user Y enters information about his car names (for example, Audi Q3), pets (two dogs and a cat), shopping preferences (say, he likes to wear clothes from Paul Smith London), travel experience (moves out of his town usually after every two weeks), job related information (say, he is a proprietor) and the like.

In an embodiment of the present disclosure, the guardians may enter the above stated information on behalf of their children (the user 102a) or any other pertinent information on the web-based platform. In another embodiment, the plurality of stake-holders 308 may enter the above stated information or any other information of the user 102a on behalf of the user 102a on the web-based platform.

In another embodiment, the user 102a may upload audios and videos of his travel experiences. For example, the users X and Y upload their videos in which they are captured while performing scuba-diving and paragliding respectively during their holiday time. In an embodiment of the present disclosure, the guardians may upload the audios and videos of their child (the user 102a) performing the adventurous sports and the like on their behalf on the web-based platform. In another embodiment, the other stake-holders of the plurality of stake-holders 308 may upload the audios and videos of the performance of the user 102a on their behalf on the web-based platform.

In an embodiment of the present disclosure, the user 102a gives ratings and/or points to himself based upon his role/performance in his company. In another embodiment, the one or more co-workers may give the ratings, the points, the comments, the messages to the user 102a based upon his performance in his work. In yet another embodiment, the other stake-holders from the plurality of stake-holders 308 may give the ratings, the points, the comments, the messages to the user 102a based upon his performance in the work, his lifestyle, preferences and the like.

Further, in an embodiment of the present disclosure, the user 102a and the plurality of stake-holders 308 may give the ratings by simply writing one or more values. In another embodiment, the user 102a and the plurality of stake-holders 308 may give the ratings by choosing from a drop down list (say, a list representing poor, average, good, very good and excellent). In yet another embodiment, the user 102a and the plurality of stake-holders 308 may give the ratings by using one or more scales. In yet another embodiment, the guardians may give comments to the user 102a based on their performances. It may be noted that various rating tools (described above) are provided to rate the user 102a based on their skills; however, those skilled in the art would appreciate that more rating tools may be provided to rate the user 102a. In an embodiment of the present disclosure, the user 102a may have the permissions to view other user's profile. Further, the user 102a may provide the ratings and the comments to each other.

Further, the data aggregation system 100 collects the information and the data described above and stores it in the database 110. Further, the data aggregation system 100 collects and maintains a repository of the collected information in the database 110. In an embodiment of the present disclosure, the user 102a may redeem the points obtained/earned by them. In an embodiment of the present disclosure, the data aggregation system 100 collects the first set of quantitative data and qualitative data from third party sources such as web, third party database, or any other source.

Further, the data aggregation system 100 enables an automatic update of the stored information in the database 110 whenever any additional information is entered by the user 102a and the stake-holders 308. In simpler terms, the database 110 gets updated at regular intervals of time with the additional information entered by the user 102a and the stake-holders 308. In an embodiment of the present disclosure, previously stored records are also maintained for future references. In other words, the database 110 is dynamic and updated on periodic basis. Further, the database 110 may store personal documents of the user (for example, his school passing certificates, his university passing certificates, identification proofs, residential proofs and the like) at one place. However, access to this information is only available to the user 102a.

For example, the user X has added a life event on the web-based platform; the same gets updated in the database 110 as well. In another example, the user Y has entered an address of his new house on the web-based platform; the same gets updated in the database 110 as well.

The data aggregation system 100 analyzes the first set of quantitative data and qualitative data uploaded and/or entered by the user 102a. In an embodiment of the present disclosure, the data aggregation system 100 analyzes performance of the user 102a in a period of time, say, in a year, last five months, last five years and the like.

The data aggregation system 100 aggregates the analyzed information at one place. The analyzed information includes analysis of the first set of quantitative data and qualitative data. Further, the data aggregation system 100 stores the aggregated information in the database 110. The aggregated information includes a collective data that influences the performance of the user 102a. In an embodiment of the present disclosure, the data aggregation system 100 aggregates performance of the user 102a over a period of time, say a month, a year, last 5 years and the like.

Further, the data aggregation system 100 generates and/or maintains pictorial representations and reports based on the analyzed and the aggregated information. The analyzed and the aggregated information can be pictorially represented as the mountains, the spiders, the graphs, the pie charts and the like. In an embodiment of the present disclosure, the pictorial representation illustrates the work performance of the user 102a in a month, year or in last five years. In yet another embodiment, the pictorial representation illustrates number of times the user 102a goes out for an adventurous tour. Further, the pictorial representations may help the user 102a and the plurality of stake-holders 308 to further give the ratings, the comments, the points and the like.

In an embodiment of the present disclosure, the data aggregation system 100 enables displaying of a set of the first set of quantitative data and qualitative data, the analyzed quantitative and qualitative information, and the aggregated quantitative and qualitative information to each of the plurality of stake-holders 308. For example, the guardians will be able to view one set of the first set of quantitative data and qualitative data, the analyzed quantitative and qualitative information, and the aggregated quantitative and qualitative information of the user 102a. The plurality of stake-holders 308 will be able to view another set of the first set of quantitative data and qualitative data, the analyzed quantitative and qualitative information and the aggregated quantitative and qualitative information.

In an embodiment of the present disclosure, the administrator of the data aggregation system 100 sets the policies and permission for accessing the information for the user 102a and the plurality of stake-holders 308. In another embodiment, the data aggregation system 100 enables the user 102a to restrict displaying and accessing of the set of information, the profile information, the analyzed and the aggregated information to the plurality of stake-holders 308. In yet another embodiment, the data aggregation system 100 enables the user 102a to restrict and/or display the set of the first set of quantitative data and qualitative data, the analyzed quantitative and qualitative information, and the aggregated quantitative and qualitative information to the plurality of stake-holders 308.

In an embodiment of the present disclosure, the data aggregation system 100 enables the user 102a to set a privacy of his account. In an embodiment of the present disclosure, the data aggregation system 100 recommends the user 102a to change his strategy for improving performance in his work. The recommendations are provided based on the analyzed information, the aggregated information, and the pictorial representations. More specifically, the data aggregation system 100 distributes the quantitative data, the qualitative data, the visualized reports, the pictorial representations and the like to the user 102a, and the plurality of stake-holders 308.

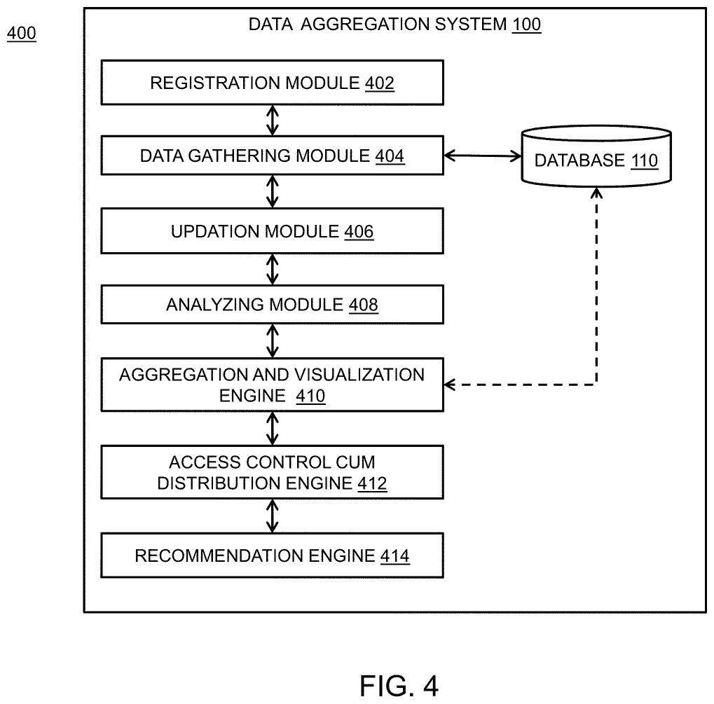

FIG. 4 illustrates a block diagram 400 of the data aggregation system 100, in accordance with various embodiments of the present disclosure. It may be noted that to explain the system elements of FIG. 4, references will be made to the system elements of the FIG. 1. The block diagram 400 of the data aggregation system 100 includes a registration module 402, a data gathering module 404, an update module 406, an analyzing module 408, an aggregation and visualization engine 410, an access control cum distribution engine 412, and a recommendation engine 414. Further, the data gathering module 404 is associated with the database 110.

The registration module 402 enables the registration of the users 102a-c on the web-based platform and/or application. Further, the registration module 402 enables registration of each of the stake-holders 112 on the web-based platform and/or application. The data gathering module 404 collects the information entered by the users 102a-c and the plurality of stake-holders 112 and stores it in the database 110. The update module 406 enables an automatic update of the stored information in the database 110 whenever any additional information is entered by the users 102a-c and the stake-holders 112. The analyzing module 408 analyzes both the first set of quantitative data and qualitative data and the second set of quantitative data and qualitative data uploaded and/or entered by each of the users 102a-c and the stake-holders 112. The aggregation and visualization engine 410 aggregates the analyzed information at one place. Further, the aggregation and visualization engine 410 generates and/or maintains pictorial representations and reports based on the analyzed and the aggregated information. The access control cum distribution engine 412 enables displaying of a set of first set of quantitative data and qualitative data and the second set of quantitative data and qualitative data, the analyzed quantitative and qualitative information, and the aggregated quantitative and qualitative information to each of the plurality of stake-holders 112. Further, the access control cum distribution engine 412 enables each of the users 102a-c to set a privacy of their respective accounts. The recommendation engine 414 recommends the plurality of stake-holders 112 to provide better opportunities to each of the users 102a-c.

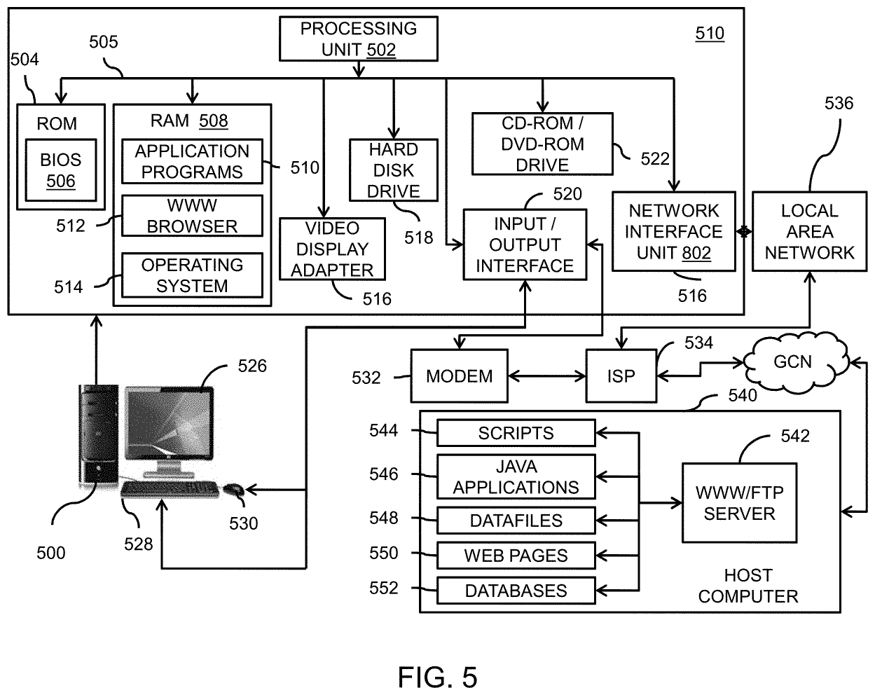

FIG. 5 provides a brief general description of a suitable computing environment in which the disclosure can be implemented. Referring now to FIG. 5, an illustrative environment includes a personal computer 500. The personal computer 500 includes a processing unit 502, a system memory, including read only memory (ROM) 504 and random access memory (RAM) 508, and a system bus 505 that couples the system memory to the processing unit 502. The read only memory (ROM) 504 includes a basic input/output system 506 (BIOS). The basic input/output system 506 contains the basic routines which transfers information between elements within the personal computer 500. Further, the personal computer 500 includes a hard disk drive 518 and an optical disk drive 522. The drives and their associated computer-readable media provide non-volatile storage for the personal computer 500. The non-volatile storage refers to a hard disk, a removable magnetic disk and a CD-ROM or DVD-ROM disk and other similar types of media are readable by a computer. The other type of media storage include magnetic cassettes, flash memory cards, digital video disks, Bernoulli cartridges, and the like. Further, those skilled in the art should appreciate that the personal computer 500 should be broadly interpreted to any machine or device being capable of processing inputs and outputs, calculating results, and in some ways connect to other devices. Examples of the personal computer 500 include but not limited to: personal desktop computers, laptop or notebook, and mobile devices such as iPad, iPhone, Android phone, Android tablet, Blackberry, Blackberry tablet, or any other “smart,” web-enabled cell phones, pocket computers or pocket organizers.

Further, program modules are stored in the drives and RAM 508. The program modules include an operating system 514 and one or more application programs 510. The application programs 510 can be a program for browsing the world-wide-web, such as WWW browser 512.

A user enters commands and information through a keyboard 528 and a pointing device. Examples of the pointing device include but may not be limited to a mouse 530. In addition, the control input devices include a microphone, joystick, game pad, satellite dish, scanner, or the like. These input devices are connected to the processing unit 500 via an input/output interface 520. The input/output interface 520 is coupled to the system bus, but may be connected by other interfaces, such as a game port, universal serial bus, or firewire port.

The personal computer 500 can operate in a networked environment using logical connections to one or more remote computers, such as a host computer 540. Examples of the host computer 540 can be a server, a router, a peer device or other common network node. The networking environments are commonplace in offices, enterprise-wide computer networks, intranets and the GCN.

In general, the host computer 540 includes many or all of the elements of the personal computer 500. The LAN 536 can be connected to a GCN service provider 534 (“ISP”) for access to the GCN 538. In this manner, WWW browser 512 connects to host computer 540 through LAN 536, ISP 534, and the GCN 538. The personal computer 500 is connected to the LAN 536 through a network interface unit 524 when used in a LAN networking environment. The personal computer 500 include a modem 532 or other means for establishing communications through the GCN service provider 534 to the GCN When used in a WAN networking environment. The modem 532 can be internal or external and is connected to the system bus 505 via the input/output interface 520.

In general, the operating system 514 controls the operation of the personal computer 500, including input/output operations. A display monitor 526 or other type of display device is also connected to the system bus 505 via an interface, such as a video display adapter 516. In addition to the monitor, personal computers typically include other peripheral output devices (not shown), such as speakers or printers. The personal computer 500 may be capable of displaying a graphical user interface on monitor 526.

The disclosure can be used in conjunction with Microsoft Corporation's “Windows 98” operating system and a WWW browser 512, such as Microsoft Corporation's GCN EXPLORER, Netscape Corporation's GCN NAVIGATOR, or Mozilla Corporation's GCN

FIREFOX, operating under this operating system. Those skilled in the art would appreciate that the disclosure can be implemented for use in other operating systems, such as Microsoft Corporation's “WINDOWS 3.1,” “WINDOWS 95”, “WINDOWS NT”, “WINDOWS 2000”, “WINDOWS XP”, “WINDOWS VISTA”, and “WINDOWS 7” operating systems, IBM Corporation's “OS/2” operating system, SunSoft's “SOLARIS” operating system used in workstations manufactured by Sun Microsystems, and the operating systems used in “MACINTOSH” computers manufactured by Apple Computer, Inc.

In general, the host computer 540 is also connected to the GCN 538, and contains components similar to those contained in personal computer 500. In addition, the host computer 540 can execute an application program for receiving requests for WWW pages, and for serving such pages to the requestor, such as WWW server 542.

The WWW server 542 transmits WWW pages 550 comprising hyper-text markup language (“HTML”) or other markup language files, such as active server pages, to WWW browser 512. Similarly, the WWW server 542 transmits requested data files 548, such as graphical images or text information, to WWW browser 512. The WWW server has the capability to execute scripts 544, such as CGI or PERL scripts, to dynamically produce WWW pages 550 for transmission to WWW browser 512.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, and to thereby enable others skilled in the art to best utilize the present invention and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the spirit or scope of the present invention.

What is claimed:

1. A data aggregation system comprising a data gathering module, in a processor, wherein the data gathering module being configured to gather a first set of qualitative and quantitative information and a second set of qualitative and quantitative information from the plurality of users and a plurality of stake-holders;
an analyzing module, in the processor, wherein the analyzing module being configured to analyze the gathered first set of qualitative and quantitative information and the second set of qualitative and quantitative information;
an aggregation and visualization engine, in the processor, wherein the aggregation and visualization engine being configured to aggregate and visualize the analyzed first set of qualitative and quantitative information and the analyzed second set of qualitative and quantitative information; and
an access control cum distribution engine, in the processor, wherein the access control cum distribution engine being configured to enable a selective distribution of a set of the gathered first set of qualitative and quantitative information and the second set of qualitative and quantitative information, the analyzed first set of qualitative and quantitative information and the second set of qualitative and quantitative information, and the aggregated first set of qualitative and quantitative information and the second set of qualitative and quantitative information to each of the plurality of stake-holders.

2. The data aggregation system of claim 1, further comprising a registration module in the processor, wherein the registration module being configured to enable registration of the plurality of users and the plurality of stake-holders.

3. The data aggregation system of claim 1, further comprising an update module in the processor, wherein the update module being configured to enable an automatic update of a stored information in a database on encountering an additional information.

4. The data aggregation system of claim 3, wherein the additional information being entered by the plurality of users and the plurality of stake-holders.

5. The data aggregation system of claim 1, further comprising a recommendation engine in the processor, wherein the recommendation engine being configured to provide better opportunities to the plurality of users and the plurality of stake-holders.

6. The data aggregation system of claim 1, wherein the first set of qualitative and quantitative information comprises sports related information, health related information, education related information, music related information and information related to miscellaneous data pertaining to the plurality of users and the plurality of stake-holders and the second set of qualitative and quantitative information comprises animal related data.

7. The data aggregation system of claim 1, wherein the first set of qualitative and quantitative information and the second set of qualitative and quantitative information being visualized by using at least one of mountains, graphs, spiders and pie-charts.

8. The data aggregation system of claim 1, further comprising a database in the processor, wherein the database being configured to store the gathered first set of qualitative and quantitative information and the second set of qualitative and quantitative information, the analyzed first set of qualitative and quantitative information and the second set of qualitative and quantitative information, and the aggregated first set of qualitative and quantitative information and the second set of qualitative and quantitative information.

9. The data aggregation system of claim 1, wherein the access control cum distribution engine being further configured to enable each of the plurality of users to set a privacy of their respective accounts on a web-based platform.

10. A computer program product comprising a non-transitory computer readable medium storing a computer readable program, wherein the computer readable program when executed on a computer causes the computer to perform steps comprising:
gathering a first set of qualitative and quantitative information and a second set of qualitative and quantitative information from the plurality of users and a plurality of stake-holders;
analyzing the gathered first set of qualitative and quantitative information and the second set of qualitative and quantitative information;
aggregating and visualizing the analyzed first set of qualitative and quantitative information and the second set of qualitative and quantitative information; and
selectively distributing a set of the gathered first set of qualitative and quantitative information and the second set of qualitative and quantitative information, the analyzed first set of qualitative and quantitative information and the second set of qualitative and quantitative information, and the aggregated first set of qualitative and quantitative information and the second set of qualitative and quantitative information to each of the plurality of stake-holders.

11. The computer program product of claim 10, wherein the computer readable program when executed on the computer causes the computer to perform a further step of enabling registration of the plurality of users and the plurality of stake-holders.

12. The computer program product of claim 10, wherein the computer readable program when executed on the computer causes the computer to perform a further step of enabling an automatic update of stored information in a database on encountering an additional information.

13. The computer program product of claim 10, wherein the computer readable program when executed on the computer causes the computer to perform a further step of providing better opportunities to the plurality of users and the plurality of stake-holders.

14. The computer program product of claim 10, wherein the computer readable program when executed on the computer causes the computer to perform a further step of storing the gathered first set of qualitative and quantitative information and the second set of qualitative and quantitative information, the analyzed first set of qualitative and quantitative information and the second set of qualitative and quantitative information, and the aggregated first set of qualitative and quantitative information and the second set of qualitative and quantitative information.

15. The computer program product of claim 10, wherein the computer readable program when executed on the computer causes the computer to perform a further step of enabling each of the plurality of users to set a privacy of their respective accounts on a web-based platform.

16. The computer program product of claim 10, wherein the computer readable program when executed on the computer causes the computer to perform a further step of enabling the plurality of stake-holders to provide specific comments and suggestions to the plurality of users.

17. The computer program product of claim 10, wherein the plurality of stake-holders comprises one or more individuals associated with the plurality of users.

18. The computer program product of claim 10, wherein the computer readable program when executed on the computer causes the computer to perform a further step of providing a chance to the plurality of users to earn profits based on their ratings.

19. The computer program product of claim 10, wherein the computer readable program when executed on the computer causes the computer to perform a further step of providing a feedback to the plurality of users and the plurality of stake-holders.

20. The computer program product of claim 10, wherein the computer readable program when executed on the computer causes the computer to perform a further step of providing parameters for measuring strengths, weakness and progress of each of the plurality of users and the plurality of stake-holders.
